(19) United States
(12) Patent Application Publication
(10) Pub. No.: US 2007/0113295 A1
(43) Pub. Date: May 17, 2007

(54) Gene blocking method

(75) Inventors: Eric H. Davidson, Pasadena, CA (US); Joel Smith, Lacanada, CA (US)

Correspondence Address: FISH & NEAVE IP GROUP;ROPES & GRAY LLP, ONE INTERNATIONAL PLACE, BOSTON, MA, 02110-2624, US

(73) Assignee: California Institute of Technology, Pasadena, CA (US)

(21) Appl. No.: 11/526,303

(22) Filed: Sep. 25, 2006

Related U.S. Application Data
(60) Provisional application No. 60/720,211, filed on Sep. 23, 2005.

(51) Int. Cl. A01K 67/027; C12N 5/06

(52) U.S. Cl. 800/8; 800/14; 435/455; 435/325

(57) ABSTRACT

The invention relates to methods of regulating gene expression, e.g., to turn on or off, or up or down gene expression in an organism when and where desired, without the toxic side effects usually associated with other contemporary methods, such as those toxic side effects caused by introducing compounds that do not naturally occur in life.

### REFERENCE TO RELATED APPLICATION

This application claims the benefit of the filing date under 35 U.S.C. § 119(e) of U.S. Provisional Application U.S. Ser. No. 60/720,211, filed on Sep. 23, 2005. The entire teachings of the referenced application are incorporated herein by reference.

### GOVERNMENT SUPPORT

Work described herein was funded, in whole or in part, by Grant No. DE-FG02-03ER63584/T-103017, from the U.S. Department of Energy. The United States government has certain rights in the invention.

### BACKGROUND OF THE INVENTION

Gene expression defines the parts and stages of a living organism. The development of an embryo, the genesis and progression of a pathological condition, or the simple progression of a cell through the cell cycle all involves regulation or de-regulation of one or more genes of an organism. Thus it is always desirable to be able to regulate the expression of any desired gene(s), either in an adult organism (including all post-natal stages animal) or in a developing embryo, preferably in a developmental-specific and/or tissue-specific manner.

One way to regulate gene expression, aside from targeting DNA (e.g., various transgenic technologies) or protein (e.g., dominant negative mutant, protein degredation, translational regulation, etc.), is transcriptional regulation that modulates the transcription of mRNA. Several representative RNA transcription antagonists include antisense agents (DNA, RNA, or derivatives thereof), RNAi (RNA interference, such as small interfering RNA or siRNA, short hairpin RNA, microRNA, etc.), aptamers, ribozyme, etc, with antisense agents being the most advanced class of these RNA transcription antagonists. See Faria and Ulrich, Curr. Cancer Drug Targets 2: 355-368, 2002.

Oligos which bind to complementary RNA sequences are commonly called “antisense” oligos because they are typically used to bind the “sense” sequence of a mature cytosolic messenger RNA (mRNA). Antisense oligos have been used for identifying the function and studying the control of genes, as well as for validating prospective protein targets in drug development programmes. Such oligos also promise therapeutics for a broad range of currently intractable diseases.

Since there are usually multiple copies of accumulated mRNA in the cytosol, a relatively large amount of such antisense oligoes with relatively long half-life are generally thought to be necessary for successful down-regulation of mRNA expression in the cytosol. Thus in the early days of antisense research (the 1970s and 1980s), most of the focus was on making structural changes to improve the stability of such DNA and RNA oligos, which in their natual state have half-lives of only a few minutes in living systems. Since structural modifications generally result in decreased affinity and/or binding specificity to the target mRNA, most early efforts entailed making minimal changes in the structure of DNA or RNA oligos, in an effort to retain the oligo's ability to bind its complementary RNA sequence while resisting enzymatic degradation. Such modifications included modifying just one end of the oligo (see, e.g., Zamecnik & Stephenson, Proc Natl Acad Sci U.S.A. 75(1): 280-4, 1978), replacing one oxygen of the phosphodiester inter-subunit linkages with methyl (Miller & Ts'o, Anticancer Drug Des. 2(2): 117-28, 1987) or with sulfur or with alkylamines (Froehler & Matteucci, Nucleic Acids Res. 16(11): 4831-4839, 1988), or entirely replacing the phosphodiester linkages with carbamates (Stirchak et al., J. Organic Chemistry 52: 4202, 1987).

While these minimal structural modifications did increase resistance to degradation, oligos utilizing these conservative modifications also suffered from serious limitations. As a case in point, the most popular of the early structural types was S-DNA (phosphorothioate DNA), wherein an oxygen on the phosphate was replaced by a sulfur. This modest change improved stability (increasing the half-life from minutes for DNA oligos to hours for S-DNA oligos), while retaining good activity against targeted RNA sequences. However, continuing research with S-DNAs brought to light a host of serious problems. Because of their limited sequence specificity, numerous off-target effects and low targeting predictability, it appears that many of the biological effects generated by any given S-DNA are typically not due to inhibition of the targeted mRNA.

By the mid-1980s, there was reason to suspect that the conservative structural modifications to DNA and RNA being pursued by most antisense research groups might never be adequate for achieving optimal antisense activity, particularly for therapeutic applications. This led to attempt a radical re-design of genetic material in the hope of developing a truly optimal antisense structural type. The most notable successes from such attempts over the past 20 years are Morpholinos, developed by Summerton and Weller (see, e.g., Summerton and Weller, Antisense & Nucleic Acid Drug Development 7: 187, 1987; Summerton, Biochemica et Biophysica Acta 1489: 141, 1999; and Summerton, Letters in Peptide Science 10: 215, 2004), and Peptide Nucleic Acids (PNAs), developed in Denmark by Nelsen and Egholm.

Morpholinos constitute a radical re-design of DNA. Key structural features of morpholino includes: 1) the 5-membered deoxyribose rings of DNA are replaced by 6-membered morpholine rings; and 2) the negatively-charged inter-subunit linkages of DNA are replaced by non-ionic inter-subunit linkages. As a consequence of their backbone structural changes, morpholinos are quite stable in biological systems, allowing relatively long-term applications, such as targeting accumulated cytoplasmic mRNA. Morpholinos tend not to interact with proteins, they are thus relatively free of off-target effects.

However, morpholinos are not without problem. The most significant limitation is in delivery of such morpholinos to the target cell, especially in vivo delivery, which is a general problem for most-modified antisense oligo designs.

Until the late-1980s, most antisense experiments were carried out in cell-free test systems, where the focus was on assessing prospective structural types for directly inhibiting the function of their targeted messenger RNAs. However, as the antisense field matured and antisense experiments began to be carried out in cultured cells, careful experiments indicated that antisense oligos were ineffective in cultured cells, because most of the oligos were not getting into the cells, and those that did enter cells were only getting into endosomes/lysosomes, where they had no access to their targeted RNA sequences, which reside in the cytosol or nuclear compartment of the cell. These findings led to wide-ranging efforts to develop effective methods for delivering oligos into the cytosol or nuclear compartment of cultured cells, and as a result, effective methods are available for delivering essentially all oligo types into cultured cells, although most such delivery systems are quite complex to use, do not work well in the presence of serum, and most are relatively toxic to the cells after just two to four hours of contact.

Furthermore, since these exogenous oligoes have to be delivered from outside the cell via, for example, microinjection (as opposed to be products of the intracellular transcriptional machinery), it is extremely hard, if possible at all, to control the delivery of such oligoes in a developmental stage- and/or tissue-specific manner.

While methods for delivering antisense oligos to the cytosol or nuclear compartment of cultured cells are now fairly well developed, most of these methods appear to be ineffective in the presence of serum and/or are too toxic for use in vivo.

In addition, morpholinos are optimized for use at about 37° C. When used at much lower temperatures (such as in frog embryos at 18° C.), a few morpholinos have been reported to inhibit some non-targeted genes.

Aside from the problems delineated above, standard methods of gene knockdown are additionally limited in several respects. For example, the gene knockdown effect may not persist from the time knockdown is induced until the time of normal gene function. Second, the gene in question may have early essential or important roles thereby confounding the study of late effects. Lastly, a gene may be expressed concurrently in different tissues of the organism, such that universal down-regulation of this gene prevents further understanding of gene function restricted to a particular domain.

The problems described above prevents the realization of the full potential of gene knockdown as an effective, indeed essential, tool for understanding gene function and, by extension, many areas of biology. Therefore, there is a need to develop new methods and reagents to regulate the expression of genes, preferably in a developmental stage- and/or tissue-specific manner, without the toxic side-effects and non-specific off-target effects associated with introduction of compounds that do not naturally occur in life.

### SUMMARY OF THE INVENTION

The instant invention relates to a general system and method for controlled-regulation of gene expression. Preferably, the system and methods of the invention uses cis-regulatory elements, such as enhancers, to regulate the expression of certain nuclear blocking sequences (e.g., antisense RNA) in an induciblly-, temporally- and/or spacially-controlled manner.

Thus one aspect of the invention provides a method for inhibiting the expression of a target gene in an organism, the method comprising: (1) providing a nucleic acid construct comprising a polynucleotide sequence encoding a nuclear blocking sequence of the target gene, the polynucleotide sequence is operably linked to a cis-regulatory module which directs the temporal- and/or spacial-expression of the nuclear blocking sequence; (2) introducing the nucleic acid construct into the organism to allow the expression of the nuclear blocking sequence, thereby inhibiting the expression of the target gene in the organism.

In certain embodiments, the inhibition of expression results in alteration of at least one phenotypic trait of the organism, preferably a detectable phenotypic trait.

In certain embodiments, the nuclear blocking sequence binds in the nucleus to a portion of the target gene transcript (e.g., pre-mRNA, tRNA precursor, rRNA precursor, or other RNA transcripts), such as an exon, an intron, or a boundary between an exon and an intron (or an intron and an exon).

In certain embodiments, the target gene comprises one or more introns, and the nuclear blocking sequence inhibits splicing of the target gene transcript.

In certain embodiments, the cis-regulatory module is a regulatory sequence controlling the spacial- and/or temporal-expression of the target gene.

In certain embodiments, the cis-regulatory module is a regulatory sequence controlling the spacial- and/or temporal-expression of a second gene different from the target gene.

In certain embodiments, the nuclear blocking sequence is an antisense RNA complementary to a portion of the target gene transcript.

In certain embodiments, the antisense RNA, when bound to the portion of the target gene transcript, activates a ribonuclease.

In certain embodiments, the portion of the target gene transcript spans the upstream splice junction of the target gene.

In certain embodiments, the splice junction is an exon-intron junction or a splice donor.

In certain embodiments, the splice junction spans the first exon or the first intron.

In certain embodiments, the splice junction is an intron-exon junction or a splice acceptor.

In certain embodiments, the length of the antisense RNA is about 25-40 bases.

In certain embodiments, about half of the length of the antisense RNA is complementary to exon sequence.

In certain embodiments, the antisense RNA binds to the target gene transcript in the nucleus to inhibit splicing.

In certain embodiments, the nucleic acid construct is stably integrated into the genome of at least one cell of the organism.

In certain embodiments, no more than 100 copies of the nuclear blocking sequence is present in nucleus.

In certain embodiments, the organism is a eukaryote, such as a unicellular organism (yeast etc.), a plant, a worm, an insect, an echinoderm, a vertebrate, a fish, a bird, a reptile, an amphibian, a mammal (e.g., a rodent, a non-human primate, a human, etc.).

In certain embodiments, the organism is a cell.

In certain embodiments, the nucleic acid construct inhibits the expression of the target gene in vitro.

In certain embodiments, the nucleic acid construct inhibits the expression of the target gene in vivo.

In certain embodiments, the method further comprises providing a second polynucleotide sequence encoding a second nuclear blocking sequence of the target gene, wherein the expression of the second nuclear blocking sequence is controlled by a second cis-regulatory module.

In certain embodiments, the nuclear blocking sequence and the second nuclear blocking sequence are both antisense RNA transcripts, with one being complementary to a splice donor, and the other being complementary to a splice acceptor.

In certain embodiments, the spice donor and splice acceptor comprise the same exon or intron.

In certain embodiments, the second cis-regulatory module is the same as the cis-regulatory module.

In certain embodiments, the cis-regulatory module comprises an inducible promoter, a tissue-specific promoter, and/or a developmental stage-specific promoter.

In certain embodiments, the inducible promoter is a tetracyclin-responsive promoter.

In certain embodiments, the tetracyclin-responsive promoter is a TetON promoter, the transcription from which promoter is activated at the presence of tetracyclin (tet), doxycycline (Dox), or a tet analog.

In certain embodiments, the tetracyclin-responsive promoter is a TetOFF promoter, the transcription from which promoter is turned off at the presence of tetracyclin (tet), doxycycline (Dox), or a tet analog.

Another aspect of the invention provides a nucleic acid construct comprising a polynucleotide sequence encoding a nuclear blocking sequence of a target gene in an organism, wherein the polynucleotide sequence is operably linked to a cis-regulatory module which directs the temporal-, spacial-, and/or inducible-expression of the nuclear blocking sequence upon introducing the nucleic acid construct into the organism, and wherein the target gene comprises one or more introns, and the nuclear blocking sequence inhibits splicing of the target gene transcript.

Another aspect of the invention provides an organism comprising any of the subject nucleic acid construct.

In certain embodiments, the organism is a cell, or a non-human animal (supra).

In certain embodiments, the non-human animal is a chimera.

In certain embodiments, the non-human animal is a transgenic animal.

Another aspect of the invention provides a method for treating a gene-mediated disease, comprising introducing into an individual having the disease a subject nucleic acid construct, where the nuclear blocking sequence is specific for the gene mediating the disease.

In certain embodiments, the nuclear blocking sequence inhibits splicing of a transcript of the gene mediating the disease.

Another aspect of the invention provides a method for validating a candidate gene as a potential target for treating a disease, comprising: (1) introducing a construct according to claim 31 into a cell associated with the disease, wherein the nuclear blocking sequence is specific for the candidate gene; (2) assessing the effect of inhibiting the expression of the candidate gene on one or more disease-associated phenotypes; wherein a positive effect on at least one disease-associated phenotype is indicative that the candidate gene is a potential target for treating the disease.

In certain embodiments, the cis-regulatory module comprises an inducible promoter.

In certain embodiments, the candidate gene is over-expressed or abnormally active in disease cells or tissues.

In certain embodiments, the candidate gene is downstream of and is activated by a second gene over-expressed or abnormally active in disease cells or tissues.

In certain embodiments, the product of the candidate gene antagonizes an suppressor of a second gene over-expressed or abnormally active in disease cells or tissues.

In certain embodiments, the cell is a tissue culture cell.

In certain embodiments, the tissue culture cell is a primary cell isolated from diseased tissues, or from an established cell line derived from diseased tissues.

In certain embodiments, the cell is within diseased tissues, and step (2) comprises evaluating one or more symptoms of the disease.

In certain embodiments, the expression of the candidate gene is inducibly inhibited by the nuclear blocking sequence encoded by a subject nucleic acid construct.

In certain embodiments, the expression of the candidate gene is inducibly activated by turning down the expression of the nuclear blocking sequence encoded by a subject construct.

All embodiments of the invention are contemplated to be applicable to all aspects of the invention where applicable, and any embodiments may be combined with the other embodiments where appropriate, unless otherwise indicated.

### BRIEF DESCRIPTION OF THE DRAWINGS

FIG. 1. Shows exemplary gene-knockdown vector design. This figure illustrates the exemplary structural elements of the spatially and temporally regulated antisense construct. Starting from the left, there is the Driver Gene cis-regulatory fragment/sequence, which can be virtually any length. The two boxes represented by thick lines represent hypothetical cis-regulatory modules where the functional transcription factor binding sites would reside. Only rough knowledge of the cis-regulatory apparatus is necessary, if at all. For example, in the case of constructs inserted into certain vectors (such as the Driver Gene BAC vectors), no prior knowledge at all is needed. The Driver Gene cis-regulatory sequences control transcription (indicated by bent arrow). The universal adaptamer sequence (24 base pair in this exemplary construct, though not necessarily so limited) may be important for vector construction only, and only in certain methods such as the fusion PCR methods. It may not be present in other constructs. The antisense target sequence may be 24 bp in length, but can be longer or shorter. It may be directed against a splice junction in the Target Gene. At the end of this exemplary construct are three repeated poly-adenylation signals. Other repeat numbers are also suitable.

FIG. 2. A flow chart showing two exemplary vector construction processes: the fusion PCR method is in the top panel; and the homologous recombination in a BAC vector is in the bottom panel. Construction by fusion PCR entails parallel synthesis of a PCR-amplified “Driver Fragment” containing all relevant cis-regulatory sequences for directing spatially and temporally defined expression; and of the “Antisense Oligo,” an oligomer containing target sequence. The Antisense Oligo and the Driver Fragment may be fused together through the use of a universal adaptamer sequence on the end of each molecule, and the fusion is amplified during two rounds of PCR thermal cycling. For BAC-antisense vector construction, sequence knowledge around the Driver Gene start of transcription is needed. “Tails” of approximately 45 bp matching sequence around the start site (“recombination sequences”) are then made to flank a recombination cassette containing the antisense sequence and Kanamycin resistance gene (Kan R) for later selection. Homologous recombination takes place at these matching recombination sequences in E. coli transformed with both the BAC and the recombination cassette.

FIG. 3. Shows the assay used and important time points. S. purpuratus embryo stages and time of development in hours post-fertilization (“hpf”) are depicted relative to time of expression of the Driver Genes (Tbr and Sm30) and phenotypic assays performed. Skeletogenic lineage cells are shown in dark. Zygotic Tbr expression starts by 8-hr post-fertilization, while Sm30 expression begins around 30-hr as illustrated by the horizontal time lines. The assay for the epithelial-to-mesenchymal ingression of skeletogenic cells occurs at 20-24-hr, before the Sm30 gene becomes active. The assays for skeleton formation (array formation and mineralization) are performed at 48-hr, after Sm30 expression is turned on.

FIG. 4. Shows representative ingression assay data. The ingression of skeletogenic cells involves ingression from the epithelium into the mesenchyme. Embryos were scored for the presence of mesenchymal or epithelial GFP⁺ cells by direct observation with fluorescence microscopy. Values are percentages+/−standard deviation. A number of embryos possessed both epithelial and mesenchymal cells displaying GFP fluorescence; values in rows therefore add up to more than 100. Pictures in the bottom panel are representative samples of the phenotypes observed. Fluorescent micrographs overlay phase contrast images.

FIG. 5. Shows representative skeletonization assay data. Two aspects of skeletonization were assessed: array formation and mineralization. By this time point (48-hr), almost all embryos in the Sense Controls (top two rows) show a normal array of skeletogenic cells and mineralization. The right panel shows a phase contrast image (top) and fluorescent image (second from top) from two control embryos to illustrate the wild-type phenotype. The arrow indicates the skeleton. By contrast, embryos harboring either Sm30- or Tbr-driven antisense constructs display a high rate of abnormal array formation or complete lack thereof (see, for instance, the bottom two pictures in the right panel). Even embryos with stunted or incomplete arrays may be competent to initiate mineralization; any evidence of mineralization was scored as positive. The table shows a majority of embryos treated with antisense vector demonstrated a complete lack of mineralization, a very stark phenotype (see text below).

### DETAILED DESCRIPTION OF THE INVENTION

1. Overview

The instant invention relates to a general method and reagents for modulating (e.g., inhibiting) gene expression. More specifically, the invention relates to the use of nuclear blocking sequences (such as antisense RNA transcribed from a polynucleotide template) controlled by a cis-regulatory sequence to inhibit the nuclear processing (e.g., including, but not limited to pre-mRNA splicing) of one or more target genes in the cell nucleus of an organism (e.g., a human or a non-human animal), preferably in a tissue-specific and/or developmental stage-specific manner. The nuclear blocking sequence of the invention may be complimentary to (e.g., binds to) one or more intron-exon boundary, exon-intron boundary, exon, or intron of a target gene. The methods and reagents of the invention have broad used in medical and research settings where it is desirable to modulate the expression of one or more target gene(s).

The instant invention is partly based on the surprising discovery that nuclear blocking sequences, such as antisense RNA transcripts, are sufficient to substantially or completely inhibit nuclear processing (e.g., including, but not limited to pre-mRNA splicing) in the nucleus, even when such nuclear blocking sequences are provided at relatively low levels, despite the fact that comparable levels of antisense RNA (endogenously transcribed, or exogenously provided) might not be sufficient to antagonize the function of mature mRNA in the cytosol. While not wishing to be bound by any particular theory, unlike cytosolic mature mRNA, nuclear pre-mRNA usually does not accumulate to a relatively high level, and are generally less stable and quickly degraded/turned over. Thus by primarily targeting the processing of the relatively few copies of pre-mRNA in the nucleus, rather than blocking protein translation initiated from the numerous copies of mature mRNA in the cytosol, the invention provides a simple yet efficient means to regulate gene transcription.

Furthermore, unlike the exogenously provided modified antisense DNA or RNA oligoes or derivatives, such naturally transcribed antisense RNA may be synthesized under the control of cis-regulatory element(s), thus achieving tissue- and/or developmental stage-specific and/or inducible regulation of gene transcription.

Also unlike the exogenously provided modified antisense DNA or RNA oligoes or derivatives, such naturally transcribed antisense RNA may be easily delivered in vitro and/or in vivo to any organism, using established delivery vectors comprising the cis-regulatory element(s) and the polynucleotides encoding the nuclear blocking sequence (e.g., antisense RNA oligoes).

The entire pre-mRNA transcript of the target gene may be destructed even when the nuclear blocking sequence is complementary to a sequence anywhere within the pre-mRNA transcript (i.e., not merely at the boundary of an intron and an exon). In other words, the nuclear blocking sequence of the invention may be complimentary to (e.g., binds to) one or more intron-exon boundary, exon-intron boundary, exon, or intron of a target gene.

While not wishing to be bound by any particular theory, such destruction may be carried out by a nuclear RNA degradation event, such as by an RNase, spliceosome, or an intracellular mechanism targeting double strand RNA structures (e.g., through the host anti-viral machinery).

Thus one aspect of the invention provides a method of inhibiting the expression of a target gene in an organism, the method comprising: (1) providing a nucleic acid construct comprising a polynucleotide sequence encoding a nuclear blocking sequence of the target gene, the polynucleotide sequence is operably linked to a cis-regulatory module which directs the temporal-, spacial-, and/or inducible expression of the nuclear blocking sequence (e.g., the expression of the nuclear blocking sequence at a desired developmental stage and/or in a desired tissue, optionally inducible expression); (2) introducing the nucleic acid construct into the organism to allow the expression of the nuclear blocking sequence, thereby inhibiting the expression of the target gene in the organism.

In certain embodiments, the nuclear blocking sequence binds in the nucleus (of a cell of the organism) to a portion of a primary pre-mRNA, or the target gene transcript. The nuclear blocking sequence may inhibit the expression of the target gene by binding to the portion in the nucleus of a target cell. For example, the nuclear blocking sequence may be complimentary to (e.g., binds to) one or more sequences encoded by intron-exon boundary, exon-intron boundary, exon, or intron of a target gene.

In certain embodiments, the target gene comprises one or more introns, and the nuclear blocking sequence inhibits the splicing and/or promotes the degradation of the target gene transcript. Targeting intron, or intron-exon/exon-intron boundaries may be advantageous, in that closely related gene in the organism may have relatively diverse intron sequences, even though these genes may have highly homologues exon sequences. Thus if at least part of the nuclear blocking sequence targets intron sequences, the method could achieve high specificity in terms of down-regulating the expression of one or more closely related genes in the organism.

As used herein, “inhibit the splicing” or grammatical variations includes either reduce or completely abolish the splicing of a pre-mRNA transcript from a gene. In certain embodiments, at least about 10, 20, 30, 40, 50, 60, 70, 80, 90, 95, 99% of the splicing (as compared to the wild-type) is inhibited. The term also covers the situation where the splicing for one or more of the alternative splicing variants is inhibited, while the splicing for the other alternative splicing variants is not appreciably affected. For example, certain target genes may alternatively splicing a pre-mRNA into several different mature mRNA species, each may be translated into a different protein product. Sometimes, these alternative splicing products may even encode different proteins with little sequence homology (see, for example, the CDK inhibitors p16^{INK4A} and p19^{ARF}). By inhibiting the use of certain splice donor and/or acceptors, through the use of one or more nuclear blocking sequences, the splicing of one or more of the alternative splicing variants may be inhibited, while the splicing for other variants remain largely unaffected. This can be useful, for example, to assess the role of different splicing variants in different tissue types, at different developmental stages, and/or upon induction at a desired time in a desired tissue, etc.

“Target” or its grammatical variation refers to the fact that the nuclear blocking sequence is complementary to a strand of the “targeted” DNA sequence or an RNA transcript of the DNA.

The nucleic acid construct may be any suitable vector, such as plasmids, YACs (Yeast Artificial Chromosomes), PACs (Plasmid Artificial Chromosomes), BACs (Bacterial Artificial Chromosomes), phagemids, cosmids, various viral vectors (including adeno-, retro- or lenti-viral vectors, etc.), or other artificial chromosomes. Typically, the nucleic acid construct includes a functional transcriptional unit that effects the expression of the polynucleotide sequence in a host. The functional transcriptional unit may include one or more of: a cis-regulatory element, a basal or minimal promoter, transcription initiation sites, transcriptional termination sites (such as poly(A) termination signals, preferably 1-5 copies, such as 3 copies, and preferably in tandem), a 3′-trailer sequence, etc.

The nucleic acid construct may additionally comprise one or more of marker genes (such as eukaryotic or prokaryotic drug resistance gene for neomycin, hygromycin, puromycin, ampicillin, etc.), reporter genes (such as fluorescent proteins GFP, RFP, BFP, YFP, etc., enzymes luciferase, alkaline phosphatase, beta-galactosidase, etc.). Such reporter genes may be inserted in place of or in addition to the polynucleotide sequence encoding a nuclear blocking sequence. When inserted in place of the polynucleotide sequence, the reporter genes may help to verify the proper expression of the polynucleotide sequence in a temporally and spacially-controlled manner.

Alternatively, the marker genes or reporter genes may be present in constructs separate from the construct encoding the nuclear blocking sequence. These different constructs may be incorporated into a host genome together, frequently at the same chromosomal locations, as if they were on the same construct.

There are numerous ways to construct the subject nucleic acid constructs comprising the cis-regulatory element and the polynucleotide encoding the nuclear blocking sequence.

Merely to illustrate, in certain embodiments, a BAC may be isolated from a library, which BAC contains a target gene to be modulated in a target host organism. A subject nuclear blocking sequence can then be introduced into an exon of the target gene through, for example, in vitro recombination. As a positive control, a reporter gene (such as a fluorescent gene) may be similarly recombined into the empty vector, so that the temporal and spacial expression of the nuclear blocking sequence in the host cell or organism can be confirmed, and its extent of expression estimated.

Thus the invention also includes methods for quantitating a level of nuclear blocking sequence expression, the method comprises incorporating a nuclear blocking sequence into a reporter system, transfecting a host cell with the reporter system, and detecting expression of a reporter gene product to quantitate the level of the nuclear blocking sequence. In some embodiments the reporter system includes a firefly luciferase reporter gene or a fluorescent protein (such as GFP and its various variants).

In certain embodiments, the polynucleotide used for the in vitro recombinantion may comprise elements other than the sequence encoding the nuclear blocking sequence. For example, it may include a drug resistance gene (which is different from the ones already on the BAC vector, if any), such as Kanamycin resistant gene, so that only the recombinants will be selected. The polynuclotide sequence may be flanked by bits of sequences homologous to the sequences sourrounding the cis-regulatory element in the vector. All these different sequences can be linked together by conventional molecular biology methods, such as restriction endonucleased digestion followed by ligation, or recombinant PCR (see below).

In other embodiments, recombinant PCR may be used to assemble different parts of the construct from different sources. For example, the cis-regulatory element may be amplified out of a source by PCR or isolated as a DA fragment via restriction digestion. The polynucleotide encoding the nuclear blocking sequence may be synthesized as an oligonucleotide. Such oligonucleotide may additionally comprise adapter sequences (such as those useful for the subsequent recombinant PCR amplification) or poly(A) signal sequences or transcription termination sequences, etc. These different nucleic acid fragments can then be mixed together for recombinant PCR amplification. Optionally, the PCR product may be subcloned and/or sequenced to ensure the proper product results.

In certain embodiments, the cis-regulatory module is a regulatory sequence controlling the spacial, temporal, and/or inducible expression of the target gene, such as an enhancer and a promoter of the target gene. In this case, the nuclear blocking sequence will be transcribed in the same tissue, and at the same developmental stage as that of the target gene. As a result, the splicing of the target gene is partially or substantially antagonized by the nuclear blocking sequence in the organism.

Alternatively, the cis-regulatory module may be a regulatory sequence controlling the expression of a second gene different from the target gene. This can be particularly useful when the transcriptional regulation of the target gene is not fully understood, and/or when the cis-regulatory element(s) of the target gene has not been identified. Under these circumstances, to turn down or off target gene expression at a desired time and place, all that is necessary is a known cis-regulatory element of a second gene (related or unrelated to the target gene), which cis-regulatory element drives the expression of an operably-linked transcription unit comprising the nuclear blocking sequence for the target gene.

In certain embodiments, various inducible Pol II promoters may be part of the cis-regulatory elements used to direct the expression of the nuclear blocking sequence (e.g., antisense RNA). Exemplary inducible Pol II promoters include the tightly regulatable Tet system (either TetOn or TetOFF), and a number of other inducible expression systems known in the art and/or described herein. The tet system allows incremental and reversible induction of the nuclear blocking sequence expression in vitro and in vivo, with no or minimal leakiness in expression. Such exemplary inducible promoters are available from Invitrogen, e.g., the GeneSwitch™ or T-REx™systems; from Clontech (Palo Alto, Calif.), e.g., the TetON and TetOFF systems.

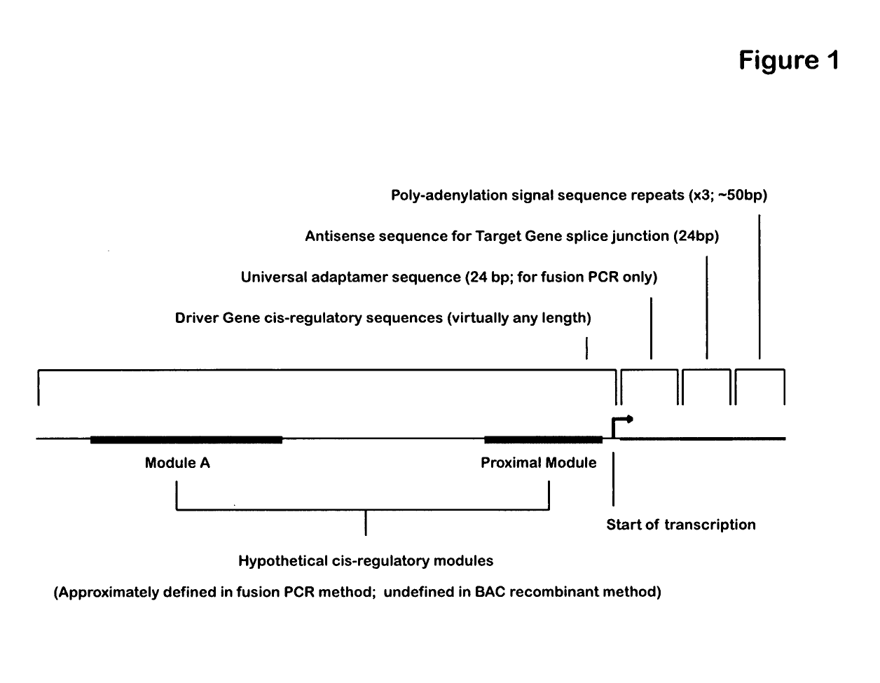

An exemplary Tet-responsive promoter is described in detail in WO 04/056964A2 (incorporated herein by reference). See, for example, FIG. 1 of WO 04/056964A2. In one construct, a Tet operator sequence (TetOp) is inserted into the promoter region of the vector. TetOp is preferably inserted between the PSE and the transcription initiation site, upstream or downstream from the TATA box. In some embodiments, the TetOp is immediately adjacent to the TATA box. The expression of the subject nuclear blocking sequence is thus under the control of tetracycline (or its derivative doxycycline, or any other tetracycline analogue). Addition of tetracycline or Dox relieves repression of the promoter by a tetracycline repressor that the host cells are also engineered to express.

In the TetOFF system, a different tet transactivator protein is expressed in the tetOFF host cell. The difference is that Tet/Dox, when bind to an activator protein, is now capable to turn off transcriptional activation. Thus such host cells expressing the activator will only activate the transcription of an encoded sequence from a TetOFF promoter in the absence of Tet or Dox.

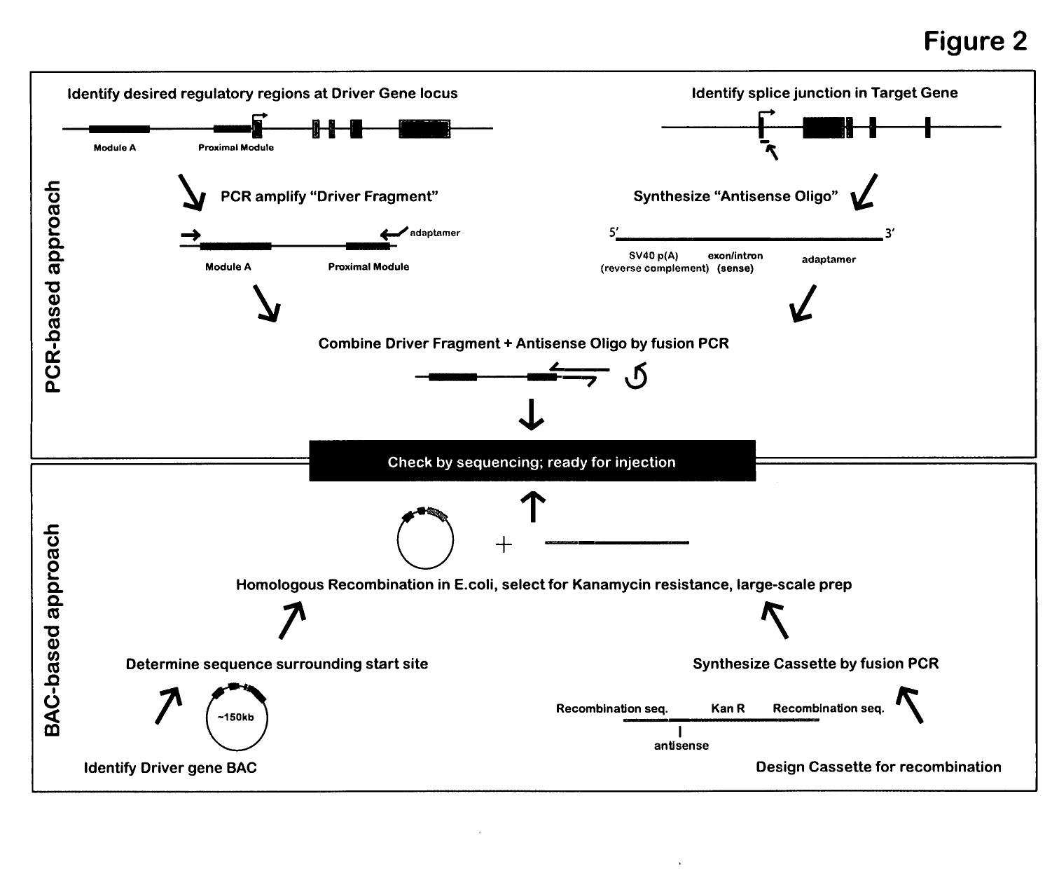

Other systems of inducible expression may also be used with the instant constructs and methods. For example, an alternative inducible promoter is a lac operator system, as illustrated in FIG. 2A of WO 04/056964 A2 (incorporated by reference). Briefly, a Lac operator sequence (LacO) is inserted into the promoter region. The LacO is preferably inserted between the PSE and the transcription initiation site, upstream or downstream of the TATA box. In some embodiments, the LacO is immediately adjacent to the TATA box. The expression of the nuclear blocking sequence is thus under the control of IPTG (or any analogue thereof). Addition of IPTG relieves repression of the promoter by a Lac repressor (i.e., the LacI protein) that the host cells are also engineered to express. Since the Lac repressor is derived from bacteria, its coding sequence may be optionally modified to adapt to the codon usage by mammalian transcriptional systems and to prevent methylation. In some embodiments, the host cells comprise (i) a first expression construct containing a gene encoding a Lac repressor operably linked to a first promoter, such as any tissue or cell type specific promoter or any general promoter, and (ii) a second expression construct containing the nuclear blocking sequence-encoding sequence, operably linked to a second promoter that is regulated by the Lac repressor and IPTG. Administration of IPTG results in expression of nuclear blocking sequence in a manner dictated by the tissue specificity of the first promoter.

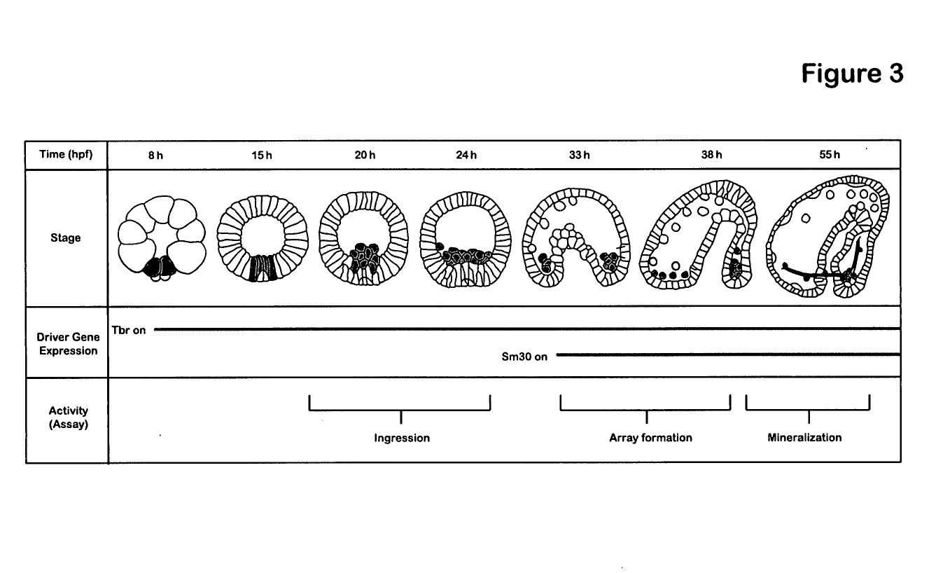

Yet another inducible system, a LoxP-stop-LoxP system, is illustrated in FIGS. 3A-3E of WO 04/056964 A2 (incorporated by reference). The vector of that system contains a LoxP-Stop-LoxP cassette before the hairpin or within the loop of a hairpin. Any suitable stop sequence for the promoter can be used in the cassette. One version of the LoxP Stop-LoxP system for Pol II is described in, e.g., Wagner et al., Nucleic Acids Research 25:4323-4330, 1997. The “Stop” sequences (such as the one described in Wagner, sierra, or a run of five or more T nucleotides) in the cassette prevent the RNA polymerase III from extending an RNA transcript beyond the cassette. Upon introduction of a Cre recombinase, however, the LoxP sites in the cassette recombine, removing the Stop sequences and leaving a single LoxP site. Removal of the Stop sequences allows transcription to proceed through the hairpin sequence, producing a transcript that can be efficiently processed into an open-ended, interfering nuclear blocking sequence. Thus, expression of the nuclear blocking sequence is induced by addition of Cre.

In some embodiments, the host cells contain a Cre-encoding transgene under the control of a constitutive, tissue-specific promoter. As a result, the nuclear blocking sequence can only be inducibly expressed in a tissue-specific manner dictated by that promoter. Tissue-specific promoters that can be used include, without limitation: a tyrosinase promoter or a TRP2 promoter in the case of melanoma cells and melanocytes; an MMTV or WAP promoter in the case of breast cells and/or cancers; a Villin or FABP promoter in the case of intestinal cells and/or cancers; a RIP promoter in the case of pancreatic beta cells; a Keratin promoter in the case of keratinocytes; a Probasin promoter in the case of prostatic epithelium; a Nestin or GFAP promoter in the case of CNS cells and/or cancers; a Tyrosine Hydroxylase, S100 promoter or neurofilament promoter in the case of neurons; the pancreas-specific promoter described in Edlund et al., Science 230: 912-916, 1985; a Clara cell secretory protein promoter in the case of lung cancer; and an Alpha myosin promoter in the case of cardiac cells.

Cre expression also can be controlled in a temporal manner, e.g., by using an inducible promoter, or a promoter that is temporally restricted during development such as Pax3 or Protein O (neural crest), Hoxal (floorplate and notochord), Hoxb6 (extraembryonic mesoderm, lateral plate and limb mesoderm and midbrain-hindbrain junction), Nestin (neuronal lineage), GFAP (astrocyte lineage), Lck (immature thymocytes). Temporal control also can be achieved by using an inducible form of Cre. For example, one can use a small molecule controllable Cre fusion, for example a fusion of the Cre protein and the estrogen receptor (ER) or with the progesterone receptor (PR). Tamoxifen or RU486 allow the Cre-ER or Cre-PR fusion, respectively, to enter the nucleus and recombine the LoxP sites, removing the LoxP Stop cassette. Mutated versions of either receptor may also be used. For example, a mutant Cre-PR fusion protein may bind RU486 but not progesterone. Other exemplary Cre fusions are a fusion of the Cre protein and the glucocorticoid receptor (GR). Natural GR ligands include corticosterone, cortisol, and aldosterone. Mutant versions of the GR receptor, which respond to, e.g., dexamethasone, triamcinolone acetonide, and/or RU38486, may also be fused to the Cre protein.

In certain embodiments, additional transcription units may be present 3′ to the first nuclear blocking sequence. For example, an internal ribosomal entry site (IRES) may be positioned downstream of the first nuclear blocking sequence insert, the transcription of which is under the control of a second promoter, such as the PGK promoter. The IRES sequence may be used to direct the expression of an operably linked second gene, such as a reporter gene (e.g., a fluorescent protein such as GFP, BFP, YFP, etc., an enzyme such as luciferase (Promega), etc.). The reporter gene may serve as an indication of infection/transfection, and the efficiency and/or amount of mRNA transcription of the nuclear blocking sequence—IRES—reporter cassette/insert. Optionally, one or more selectable markers (such as puromycin resistance gene, neomycin resistance gene, hygromycin resistance gene, zeocin resistance gene, etc.) may also be present on the same vector, and are under the transcriptional control of the second promoter. Such markers may be useful for selecting stable integration of the vector into a host cell genome.

A second transcription unit encoding a second nuclear blocking sequence may also be inserted in place of the reporter gene. This may be useful where the expression of two or more target genes are to be modulated, or where two or more nuclear blocking sequences are to be used to for the same target gene (such as targeting different regions of the target gene, or targeting different alternative splicing variants, etc.).

In certain embodiments, the cis-regulatory elements for the different nuclear blocking sequences may be the same or different.

Thus in certain embodiments, the method further comprises providing a second polynucleotide sequence encoding a second nuclear blocking sequence, which may be for the same or different target gene, wherein the expression of the second nuclear blocking sequence is controlled by a second cis-regulatory module. The second cis-regulatory module may be the same or different from the first cis-regulatory module. This is useful, for example, in situations where blocking only one splice junction of the pre-mRNA may force the splicing machinery to use a cryptic alternative splicing site on the pre-mRNA. By providing a second, independent splicing blocking sequence, the chance of having a functional alternative splicing product is greatly reduced, if not completely eliminated. This may also be useful in situations where inhibiting the splicing of one or more (but not all) of the alternative splicing variants is desired.

For example, the first and second nuclear blocking sequences may both be antisense RNA transcripts, with one being complementary to a splice donor, and the other being complementary to a splice acceptor of the same exon or intron. Preferably, the two blocking sequences blocks the splice donor and the splice acceptor of the first intron, respectively.

Alternatively, both blocking sequences may be specific for splicing donors or splicing acceptors if there are more than one intron.

Certain exemplary vectors useful for expressing the subject nuclear blocking sequences are discussed in a separate section below. The invention encompasses the nucleotide sequence of such vectors as well as variants thereof.

In certain embodiments, expression of the subject nuclear blocking sequence may be under the control of a tissue specific promoter, such as a promoter that is specific for: liver, pancreas (exocrine or endocrine portions), spleen, esophagus, stomach, large or small intestine, colon, GI tract, heart, lung, kidney, thymus, parathyroid, pineal gland, pituitary gland, mammary gland, salivary gland, ovary, uterus, cervix (e.g., neck portion), prostate, testis, germ cell, ear, eye, brain, retina, cerebellum, cerebrum, PNS or CNS, placenta, adrenal cortex or medulla, skin, lymph node, muscle, fat, bone, cartilage, synovium, bone marrow, epithelial, endothelial, vascular, nervous tissues, etc. The tissue specific promoter may also be specific for certain disease tissues, such as cancers. See Fukazawa et al., Cancer Research 64: 363-369, 2004 (incorporated herein by reference).

Any tissue specific promoters may be used in the instant invention. Merely to illustrate, Chen et al. (Nucleic Acid Research, Vol. 34, database issue, pages D104-D107, 2006) described TiProD, the Tissue-specific Promoter Database (incorporated herein by reference). Specifically, TiProD is a database of human promoter sequences for which some functional features are known. It allows a user to query individual promoters and the expression pattern they mediate, gene expression signatures of individual tissues, and to retrieve sets of promoters according to their tissue-specific activity or according to individual Gene Ontology terms the corresponding genes are assigned to. The database have defined a measure for tissue-specificity that allows the user to discriminate between ubiquitously and specifically expressed genes. The database is accessible at tiprod.cbi.pku dot edu.cn:8080/index.html. It covers most (if not all) the tissues described above.

Tissue-specific or developmental-stage specific promoter may be advantageous in certain embodiments, because these cis-regulatory elements are generally less “leaky” than the inducible promoters, or non-leaky at all. This is so partly because of the force of natural selection.

In certain embodiments, the nuclear blocking sequence is an antisense RNA complementary to a portion of the target gene transcript. The antisense RNA, when transcribed in the nucleus, binds to the portion of the target gene transcript and prevents the target pre-mRNA from being further processed into mature mRNA. While not wishing to be bound by any particular theory, the antisense RNA/target pre-mRNA complex may be a substrate for a ribonuclease (RNases), such as an exo- and/or endoribonucleases, and is subject to degradation.

In certain embodiments, the portion of the target gene transcript spans the upstream splice junction of the target gene. The splice junction may be an exon-intron junction or a “splice donor.” Alternatively, the splice junction is an intron-exon junction or a “splice acceptor.” Preferably, the splice junction spans the first exon or the first intron.

In certain embodiments, the antisense RNA is at least about 10, 12, 14, 16, 20, 25, 30, 35, 40, 50, 75, 100 bases or more.

In certain embodiments, the antisense RNA is no more than about 200, 100, 90, 80, 70, 60, 50, 40, 30, or 25 bases.

In certain embodiments, the antisense RNA is about 25-40 bases, or about 20-50 based, or about 14-60 bases in length.

In certain embodiments, about half of the length of the antisense RNA is complementary to exon sequence, while the other half complementary to intron sequence. In other embodiments, about 35-65%, or about 40-60% of the length of the antisense RNA is complementary to exon sequence.

In certain embodiments, depending on the specific vector chosen, the nucleic acid construct is stably integrated into the genome of at least one cell of the organism. Alternatively, the nucleic acid may be stably maintained in the host cell as an extra-chromosomal genetic material, which may or may not be “inherited” by the daughter cells.

In certain embodiments, the nucleic acid construct synthesizes the blocking sequence in the nucleus, which accumulates no more than 500 copies, 300 copies, 200 copies, 100 copies, 75 copies, 50 copies, 30 copies, 20 copies, 10-copies or fewer of the nuclear blocking sequence in the nucleus at any time.

The invention applies to any eukaryotic organism, unicellular or multicellular, so long as a proper vector for delivering the nucleic acid construct is available in that organism. The eukaryotic organism may be a plant, a unicellular organism (such as a yeast), an animal including a human, a non-human primate or mammal, a rodent (mouse, rat, hamster, rabbit, etc.), a domestic animal (cattle, sheep, goat, horse, pig, cat, dog, etc.), a species of fish (e.g., zebra fish), an echinoderm (e.g., sea urchin), an insect (e.g., Drosophila), a worm (such as C. elegans), etc.

In certain embodiments, the organism is not a C. elegans or other nematodes (worms).

In certain embodiments, the organism is a single cell (e.g., a unicellular organism or a single cell of a multicellular organism).

In certain embodiments, the nuclear blocking sequence comprises no morpholino-substitutions, PNA, phosphorothioate, or any other not naturally-occurring modifications to the base, phosphodiester linkage, or sugar ring of DNA or RNA.

A Morpholino oligo specifically binds to its selected target site to block access of cell components to that target site. A Morpholino oligo is radically different from natural nucleic acids, with morpholine rings replacing the ribose or deoxyribose sugar moieties and non-ionic phosphorodiamidate linkages replacing the anionic phosphates of DNA and RNA. Each morpholine ring suitably positions one of the standard DNA bases (A,C,G,T), so that a 25-base Morpholino oligo strongly and specifically binds to its complementary 25-base target site in a strand of RNA via Watson-Crick pairing. Because the backbone of the Morpholino oligo is not recognized by any cellular enzymes or signaling proteins, it is completely stable to nucleases and does not trigger an innate immune response through the toll-like receptors.

Though Morpholino oligoes are much more soluble than other non-ionic structural types (such as PNAs), some Morpholinos with high G content (>30%) do have limited solubility. Morpholinos tagged with our red lissamine fluor sometimes also have limited solubility. Long-term storage at 4° C. can also cause slow precipitation of Morpholinos. Keeping the concentration of Morpholino stock solution above 1 mM may also cause solubility problems. Having stretches of four or more contiguous G may also render a morpholino oligo insoluble in water. In addition, morpholinos are optimized for use at about 37° C.; when used at much lower temperatures (such as in frog embryos at 18° C.), a few morpholinos have been reported to inhibit some non-targeted genes. And certainly, one of the biggest problem with the morpholino oligoes is effective delivery, i.e., it cannot be synthesized by the target cell at a high concentration in the nucleus.

In certain embodiments, the nucleic acid construct inhibits the expression of the target gene in vitro. In another embodiment, the nucleic acid construct inhibits the expression of the target gene in vivo.

In certain embodiments, the cis-regulatory module may comprise an inducible promoter, a tissue-specific promoter, and/or a developmental stage-specific promoter (supra).

For example, the inducible promoter may be a tetracyclin-responsive promoter, such as a TetON promoter, the transcription from which promoter is activated at the presence of tetracyclin (tet), doxycycline (Dox), or a tet analog. Alternatively, the tetracyclin-responsive promoter may be a TetOFF promoter, the transcription from which promoter is turned off at the presence of tetracyclin (tet), doxycycline (Dox), or a tet analog.

Another aspect of the invention provides a nucleic acid construct comprising a polynucleotide sequence encoding a nuclear blocking sequence of a target gene in an organism, wherein the polynucleotide sequence is operably linked to a cis-regulatory module which directs the temporal-, spacial-, and/or inducible-expression of the nuclear blocking sequence upon introducing the nucleic acid construct into the organism, and wherein the target gene comprises one or more introns, and the nuclear blocking sequence inhibits splicing of the target gene transcript.

Another aspect of the invention provides an organism comprising the subject nucleic acid construct. For example, the organism may be a cell (supra), or may be a non-human animal (supra). In certain embodiments, the non-human animal is a chimera (e.g., only certain cells of the organism comprises the subject nucleic acid constructs encoding the nuclear blocking sequence). In other embodiments, the non-human animal is a transgenic animal harboring a germ-line transmission of the subject nucleic acid construct.

Another aspect of the invention provides a method for treating a gene-mediated disease, comprising introducing into an individual having the disease a subject nucleic acid construct (supra), where the nuclear blocking sequence is specific for the gene mediating the disease.

In certain embodiments, the nuclear blocking sequence inhibits splicing of a transcript of the gene mediating the disease.

Another aspect of the invention provides a method of validating a candidate gene as a potential target for treating a disease, comprising: (1) introducing a subject construct into a cell associated with the disease, wherein the nuclear blocking sequence is specific for the candidate gene; (2) assessing the effect of inhibiting the expression of the candidate gene on one or more disease-associated phenotypes; wherein a positive effect on at least one disease-associated phenotype is indicative that the candidate gene is a potential target for treating the disease.

In certain embodiments, the cis-regulatory module comprises an inducible promoter, such that the nuclear blocking sequence can be induced to express or not to express at a desired time or place.

According to this aspect of the invention, the subject construct can be used to knock down the expression of a target gene, such as a target gene that is over-expressed or abnormally active in disease cells or tissues, or a target gene that is downstream of and is activated by a second gene over-expressed or abnormally active in disease cells or tissues, or a target gene that antagonizes an suppressor of a second gene over-expressed or abnormally active in disease cells or tissues. If eliminating the function of such a target gene slows down or even reverses disease progression, the target gene is a valid target for future intervention, such as by establishing a large scale high throughout drug screening assay for identifying small molecule inhibitors of the target gene, etc.

In certain embodiments, the cell may be a tissue culture cell, such as a primary cell isolated from diseased tissues, or from an established cell line derived from diseased tissues. In other embodiments, the cell may be within diseased tissues, and step (2) above comprises evaluating one or more symptoms of the disease.

In certain embodiments, the expression of the candidate gene may be inducibly inhibited by the nuclear blocking sequence encoded by the subject construct, such as a pre-determined time. This can be useful, for example, to assess the effect of knocking down the expression of a target gene (such as an oncogene) once a disease (such as cancer) has already been initiated.

In certain other embodiments, the expression of the candidate gene is inducibly activated by turning down the expression of the nuclear blocking sequence encoded by the subject construct. This can be useful, for example, to assess the effect of turning on certain genes (such as tumor suppressor genes) in a disease tissue (such as cancer tissue that has lost both copies of the tumor suppressor gene) for assessing the efficacy of, for example, restoring gene function by gene therapy.

These and other aspects of the invention are more fully described in the sections below, including in the non-limiting examples.

2. Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

As used in this specification and the appended claims, the singular forms “a”, “an,” and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

As used herein “cis-regulatory modules,” or “transcriptional regulatory sequence,” including grammatical variations thereof, are the specific DNA sequences that directly regulate expression of a given gene. It is a generic term used throughout the specification to refer to DNA sequences, such as initiation signals, enhancers, and promoters, which induce or control transcription of protein coding sequences with which they are operably linked. In preferred embodiments, transcription of a gene is under the control of a promoter sequence (or other transcriptional regulatory sequence) which controls the expression of the gene in a cell-type in which expression is intended, and/or at a developmental stage (or any desired growth period) when expression is intended. It will also be understood that the gene can be under the control of transcriptional regulatory sequences which are the same or which are different from those sequences which control transcription of a naturally-occurring form of the gene.

As used herein “indel,” including grammatical variations thereof, means insertion and/or deletion of nucleotide sequences.

As used herein “informative alignment,” including grammatical variations thereof, means the appropriateness of the relative positioning of sequences that allows firm conclusions about the structure of conserved patterns to be drawn such that one region of sequence is favored over another. For example, regions with many insertions and deletions in the alignment are less informative.

As used herein, “genomic target site clusters,” means sites along a given genome where transcription factors bind.

As used herein, “SNP/indel intensity parameter” means the measure of SNP/indels used in a window to define similarity and statistical significance between aligned sequences. In a related aspect, such windows can be about 10 bp to about 20 bp, about 20 bp to about 30 bp, about 30 bp to about 40 bp, or about 40 bp to about 50 bp. In another related aspect, sequence similarity or homology is about 70%, about 75%, about 80%, about 85%, about 90%, or about 95%.

As used herein, the term “nucleic acid” refers to polynucleotides such as ribonucleic acid (RNA), and, where appropriate, deoxyribonucleic acid (DNA). The term should also be understood to include single-stranded (such as sense or antisense) and double-stranded polynucleotides, and, as applicable to the embodiment being described, equivalents, analogs of either RNA or DNA made from nucleotide analogs.

As used herein, the term “gene” refers to a nucleic acid comprising an open reading frame encoding a gene product such as protein or RNA (e.g., rRNA, tRNA, etc.), including both exon and (optionally) intron sequences.

The term “intron” refers to a DNA sequence present in a given gene which is not present in mature messenger RNA (mRNA), and is not translated into protein. An intron is generally found between exons.

As used herein, the term “transfection” means the introduction of a nucleic acid, e.g., an expression vector, into a recipient cell by nucleic acid-mediated gene transfer.

“Transformation,” as used herein, refers to a process in which a cell's genotype is changed as a result of the cellular uptake of exogenous DNA or RNA, and, for example, the transformed cell expresses a polynucleotide encoded by an exogenous construct, or where anti-sense expression occurs, from the transferred gene, the expression of a naturally-occurring form of a target gene for the antisense construct is disrupted.

As used herein, the term “vector” refers to a nucleic acid molecule capable of transporting another nucleic acid to which it has been linked. One type of vector is an episome, i.e., a nucleic acid capable of extra-chromosomal replication. Some vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably, as the plasmid is the most commonly used form of vector. However, the invention is intended to include such other forms of expression vectors which serve equivalent functions and which become known in the art subsequently hereto, including PAC, BAC, viral-based vectors, or artificial chromosome, etc.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue. The term also covers so-called “leaky” promoters, which regulate expression of a selected DNA primarily in one tissue, but cause expression in other tissues as well, although may be to a lesser degree.

As used herein, a “transgenic animal” is any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or sequence which may be aligned for purposes of comparison. When a position in the compared sequence is occupied by the same base or amino acid, then the molecules are homologous at that position. A degree of homology between sequences is a function of the number of matching or homologous positions shared by the sequences. In certain embodiments, the transgenic animal is not a C. elegans or other nematodes (worms).

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a first polypeptide with a second amino acid sequence defining a domain foreign to and not substantially homologous with any domain of the first polypeptide. A chimeric protein may present a foreign domain which is found (albeit in a different protein) in an organism which also expresses the first protein, or it may be an “interspecies,” “intergenic,” etc., fusion of protein structures expressed by different kinds of organisms.

The term “isolated” as also used herein with respect to nucleic acids, such as DNA or RNA, refers to molecules separated from other DNAs. or RNAs, respectively, that are present in the natural source of the macromolecule. For example, an isolated nucleic acid encoding one of the subject target gene preferably includes no more than 10 kilobases (kb) of nucleic acid sequence which naturally immediately flanks that particular gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

3. Identification of Cis-Regulatory Modules

The instant invention utilizes cis-regulatory modules to control gene expression in a temporarily- and/or spacially-specific manner. US-2006-0141513 A1 describes in detail a method of identifying various such cis-regulatory modules for use in the instant invention. The entire teaching of US-2006-0141513 A1 are incorporated herein by reference.

Specifically, US-2006-0141513 A1 relates to identification of cis-regulatory modules in genomes by comparing selected interspecific genome sequences using statistical targeting of putative patches, which patches contain suppressed indels and SNPs in regions within such patches when compared to flanking sequences.

In one embodiment, a method of identifying a cis-regulatory module is provided, the method including: (1) determining sequence similarities significantly greater than random expectation on selected genome sequences from two or more closely related species in sequences that lie outside of protein coding regions, (2) sorting the similarities for conserved patches of single nucleotide polymorphisms (SNPs) and insertion/deletions (indels), (3) constructing a computational map of SNPs/indels, where the SNPs/indels have occurrence rates within the patches which are suppressed when compared to flanking sequences, (4) computing a moving window snp/indel intensity parameter based on the patches, and moving the window across a query sequence, where a putative cis-regulatory module is identified if a region in the query sequence significantly matches the window parameter.

In one aspect, the computational map is from one or more closely related primate species, including where the primate is an ape, monkey, or human. In a further aspect, the method includes comparing the cis-regulatory modules based on the primate derived computational map to select genome sequences from non-primates and predicting cis-regulatory modules in the non-primate sequences.

In one aspect, the flanking regions comprise large indels having a length of at least 6-10 nucleotides. In another aspect, the suppressed occurrence rate within the patches for SNPs exhibits a decrease in frequency of about 30% to about 50% when compared to flanking sequences.

In another aspect, the method includes calculating the ratio of indels of differing lengths in transcriptionally active sequences versus flanking sequences, wherein the length of the indels is about 1 to 5 nucleotides, about 6 to 10 nucleotides, about 11-15 nucleotides, about 16 to 20 nucleotides, or greater than about 21 nucleotides. In a related aspect, the ratio of indels of about 6 to 10 nucleotides is between about 0 to about 0.7.

In another aspect, determining sequence similarity includes using a computer algorithm to compare aligned sequences.

US-2006-0141513 A1 also provides a computational map generated by the method; a library of genomic target site clusters including putative cis-regulatory modules identified by the method; a computer readable medium having computer-executable instructions for performing the method, etc.

The following subsections describe in more detail certain aspects of the cis-regulatory module identification method.

In one aspect, where the transcription factor target sites are not known in advance, the method provides an interspecific sequence comparison method for physically identifying putative cis-regulatory modules in the intronic or intergenic DNA sequence of given animal genes. As has long seemed reasonable to assume on the grounds that they are functionally essential, these key regulatory units are evolutionarily conserved relative to flanking sequence.

The DNA of functional cis-regulatory modules displays extensive sequence conservation in comparison of genomes from closely species. Patches of sequence that are several hundred base pairs in length within these modules are often seen to be 80-95% identical, although the flanking sequences cannot even be aligned (e.g., due to a high number of indels).

In one aspect, percent sequence identity may be calculated using computer programs or direct sequence comparison. A plurality of homology search algorithms may be used to determine optimal alignment of sequences. These include the local homology algorithm of Smith & Waterman, Adv Appl Math (1981) 2:482, the homology alignment algorithm of Needleman & Wunsch, J Mol Biol (1970) 48:443, the similarity method of Pearson & Lipman, Proc Natl Acad Sci USA (1988) 85:2444, the PSI-Blast homology algorithm of Altschul et al., Nucleic Acids Res (1997) 25:3389-402, the computerized implementations of algorithms GAP, BESTFIT, FASTA, and TFASTA included in the Wisconsin Genetics Software Package, Genetics Computer Group, 575 Science Dr., Madison, Wis.), by Hidden Markov Models (HMM, Durbin, Eddy, Krogh & Mitchison, Cambridge University Press, 1998), or EMotif/EMatrix to identify sequence motifs (Nevill-Manning et al., Proc Natl Acad Sci USA (1998) 95(11):5865-71), or by visual inspection (see generally Ausubel et al., supra). Each of the above identified algorithms and the references are herein incorporated by reference in its entirety for all purposes. These algorithms are well known to one of ordinary skill in the art of molecular biology and bioinformatics. When using any of the aforementioned algorithms, the user will define parameters for “Window”, gap penalty, and the like (e.g., the user can define the window-size, how window boundaries are determined, how gaps will be handled, and how absolute similarity and statistical significance will be indicated in program output). Practitioners of the art molecular biology with average skill will recognize these parameters (e.g., gap penalty is a scoring value to prevent large gaps from occurring in reported alignments).

Thus, as provided herein, cis-regulatory modules can be detected computationally by interspecific comparison of the sequence surrounding a gene of interest, recognized as a block of sequence that has remained relatively similar between two or more species.

Such sequences may be excised by, e.g., but not limited to, PCR, and incorporated in an expression vector. Their function can be studied by direct gene transfer methods. In one aspect, for “closely related species,” the appropriate evolutionary species distance is not so close such that unselected (i.e., “background”) sequences have not had time to diverge, but the distance is not so far that the pattern of conservation has been lost by too much divergence. In a related aspect, the evolutionary distance may range from about 1 to about 5 million years, about 5 to about 10 million years, about 10 to about 20 million years, about 20 to about 30 million years, about 20 to about 50 million years, or about 50 to about 100 million years.

At the appropriate distance, cis-regulatory modules stand out from the immediately flanking background as patches of well conserved sequence that are usually several hundred base pairs in length and terminated at their boundaries by abrupt transitions to sequence that has diverged too greatly for facile computational alignment.

The cis-regulatory modules may be defined experimentally as DNA fragments that, as a whole, faithfully recreate given developmental patterns of expression in gene transfer experiments. They consist of the target sites for the transcription factors to which they respond, plus the sequence intervening between these sites.

Although interspecific sequence comparisons may reveal cis-regulatory modules as long contiguous patches of sequence that are relatively well conserved with respect to external sequences, it is not obvious why there would be deleterious effects of sequence change outside the specific base pairs that participate directly in chemical interactions with transcription factor amino acid side chains. In three dimensional analysis of DNA-transcription factor complexes, detailed mutational studies, and “selex” assays, only a few base pairs per interaction are seen to be partially or wholly constrained, and these elements are commonly confined to short sequences typically about 6 to about 8 base pairs in length. Furthermore, for well studied examples, there is direct evidence that the actual transcription factor target sites often occupy less than half of the module length. This evidence is of several kinds, including (i) oligonucleotides mapping of all specific sites of DNA-protein interaction (see, e.g., Yuh et al., Mech Dev (1994) 47:165-186), (ii) numerous reconstruction mutation studies in which modular sequences are altered without discernable effects on function except when constrained nucleotides within target sites are changed (see, e.g., Davidson, Genomic Regulatory Systems: Development and Evolution, 2001, Academic Publishing, San Diego, Calif.; Yuh et al., Science (1998) 279:1896-1902; Yuh et al., Development (2001) 128:617-628; and Kirchhammer and Davidson, Development (1996) 122:353-348), (iii) studies on regulatory modules of which the transacting factors are known and the sites of their interaction can be recognized in the sequence (see, e.g., Arnone and Davidson, Development (1997) 124:1851-1864; and Davidson (2001)), and (iv) comparative studies on orthologous cis-regulatory modules from animals that are so distant from one another that only the transcription factor target sites are unchanged (see, e.g., Tümpel et al., Dev Biol (2002) 246:45-56; Shashikant et al., Proc Natl Acad Sci USA (1998) 95:15446-15451; Kim et al., Proc Natl Acad Sci USA (2000) 97:1655-1660; Ludwig et al., Development (1998) 125:949-958; Langeland and Carroll, Development (1993) 117:585-596; and Williams et al., Nature (1994) 368:299-305).

Though not to be bound by theory, this suggests that the target sites themselves are spaced by intervening sequences that have undergone a great deal of change during evolution. The evidence combines to exclude the idea that the observed patterns of cis-regulatory module conservation are due to functional nucleotide-by-nucleotide selection across the whole length of the module.

A mechanism that might account for what is observed is as follows. Again, not to be bound by theory, in the evolution of cis-regulatory modules, the occurrence of indels that are large enough to be likely to affect adjacent target sites might be selectively disfavored, whereas the occurrence (fixation) of single-nucleotide substitutions and small indels between transcription factor target sites is not constrained, although change within the sites themselves is, of course, constrained. It has been observed that for several cases the rate of indel accumulation in unselected sequence is sufficiently high to account for a large fraction of the total sequence change during divergence (see, e.g., Britten et al., Proc Natl Acad Sci USA (2003) 100: 4661-4665; Britten, Proc Natl Acad Sci USA (2002) 99:13633-13635; and Fujiyama et al., Science (2002) 295:131-134). Given these observations, the relative suppression within cis-regulatory modules of large indels but not of small indels or single-nucleotide changes gives the following predictions: (i) Comparison of two genomes just sufficiently distant so that nonselected sequence cannot usually be aligned will indeed reveal cis-regulatory modules as internally aligned, and thus apparently conserved patches of sequence, because the occurrence of large indels rapidly generates sequence that cannot easily be aligned, whereas, until it approaches saturation, the occurrence of single-nucleotide substitutions or small indels does not. (ii) Within these patches, the rate of occurrence of single nucleotide substitutions and of small (one or a few base pairs long) indels will be similar to the rate outside them after correcting for the fraction of the modules included in the actually constrained target site sequence. (iii) At greater evolutionary distance, as small changes accumulate, the apparent conservation of the module as a whole will disappear, because similarities of the unconstrained portions of the intramodular sequence will be lost, and only the transcription factor target sites themselves will be retained as conserved sequence elements.

That cis-regulatory modules can be effectively identified by detection of patchy interspecific sequence conservation consistent with prediction (i), is the starting point. Consistent [with prediction (iii)] is the observation that at great evolutionary distance, patchy sequence conservation of cis-regulatory modules can no longer be seen, even where gene transfer experiments reveal conserved target site function.

The requirements are (i) to ascertain sequence divergence within cis-regulatory modules that are already known experimentally to be functional, so that the comparison of sequences within and outside its boundaries is meaningful and (ii) that a species pair be used that is sufficiently close so that the genomic sequence can be unequivocally aligned both inside and outside selectively conserved features.

In one aspect, “selected genomic sequences” will be obtained for a sequenced target genome within which to search for the relevant cis-regulatory modules. For example, but not limited to, an insert that extends from the adjacent gene on the 5′-side of the gene of interest to the adjacent gene on the 3′-side, minus certain classes of sequence that are stripped out computationally, may serve as a selected genome sequence. In the case of clustered genes of the same family, e.g., Hox genes or some of the NK class homeodomain genes, certain sequences may not be excluded on the other side of the adjacent genes because of their associated functional consequences if deleted, but many genes of interest are unique, and are not found in paralogue clusters (i.e., homologous because of a gene duplication event).

The sequences stripped out are those exonic sequences encoding protein, direct simple sequences (mono-, di-, and tri-nucleotide repeats greater than 11 bp in length), and recognizable repetitive sequences. Repetitive sequences may be highly species-specific and in the absence of extensive genomic sequence data, may be difficult to recognize at the sequence level. However, one of skill in the art may modify this criterion to serve user specific requirements. For example, while BAC-end sequence resources deriving from various genome projects can provide a useable library of repeat elements for their associated species, only the higher frequency repeats are routinely identified. Again, this criterion may be modified by the user.

In a related aspect, for example, but not limited to, all sequence elements 500 bp long to all others within a genomic sequence are compared, looking for any sequence similarities significantly greater than random expectation. For example, the statistical significance of genome mapping may be determined by chi-square test of observed number of orthologs between genomic sequences and a randomly expected number, with respect to the smallest number of genes on these genomes. The random expectation can be calculated as a fraction of the number of orthologs on the genome of one of a first corresponding closely related species that would be expected to fall on the genome of a second species in the pair, assuming uniform distribution over all of the genes of the second closely related species. Alternatively, Hidden Markov Modeling may be used to determine the likelihood of an observation that is significantly greater than random expectation (e.g., see en.wikipedia dot org/wiki/Hidden_Markov_Model). Further, other means include Poisson metrics.

These similarities are then sorted for families of sequence elements ≧80% or 90% homologous. Thus, as data accumulates for each species, a log of both locally repeated sequence elements (e.g., within given genomic sequence) and globally interspersed repeated sequences (e.g., among genomes) is constructed. These may be flagged, or if identified clearly enough, stripped from the selected sequence. What remains of the selected sequence surrounding the gene of interest is then used as the search basis for putative/conserved patches. This will be the largely single copy sequences flanking the gene on either side, plus intronic sequences.

To annotate the sequences, sequencing may be searched preliminarily for sequenced genes identifiable by comparison with protein data banks (e.g., TRANSFAC transcription database, maintained at the GBF Brunschweig, Germany; GenBank, National Institutes of Health) and then analyzed by various annotation programs (e.g., modified Genotator; Sea Urchin Genome AnnotatoR (SUGAR); GLIMMERM, The Institute for Genomic Research (TIGR), and the like). Selected genome regions are identified then stripped.

In one embodiment, a method of identifying a cis-regulatory module is provided, including: (1) determining sequence similarities significantly greater than random expectation on selected genome sequences from two or more closely related species in sequences that lie outside of protein coding regions, (2) sorting the similarities for conserved patches of single nucleotide polymorphisms (SNPs) and insertion/deletions (indels), (2) constructing a computational map of SNPs/indels, where the SNPs/indels have occurrence rates within the patches which are suppressed when compared to flanking sequences, (3) computing a moving window snp/indel intensity parameter based on the patches, and (4) moving the window across a query sequence, where a putative cis-regulatory module is identified if a region in the query sequence significantly matches the window parameter. In a related aspect, a computational map generated by the disclosed method is provided.

Nucleic acids so identified can be amplified from genomic DNA using established polymerase chain reaction (PCR) techniques (see K. Mullis et al. (1986) Cold Spring Harbor Symp. Quant. Biol. 51:260; K. H. Roux (1995) PCR Methods Appl. 4:S185) in accordance with the nucleic acid sequence information provided herein.

In another aspect, alignment/predictive algorithms include, but are not limited to, BLASTN (ncbi.nlm.nih dot gov/BLAST/), FAMILY RELATIONS (FR) (family.caltech dot edu/), CLUSTAL W (Bioinformatics: A Practical Guide to the Analysis of Genes and Proteins, (2001), 2nd ed., (Baxevanis and Ouellette, eds.), Wiley-Interscience, New York, N.Y.), AMPS (Barton, Methods Enz (1990) 183:403-428), GENSCAN (Burge and Karlin, Curr Opin Struct Biol (1998) 8:346-354), PROCRUSTES (Gelfand et al., Proc Natl Acad Sci USA (1996) 93:9061-9066), GeneParser (Snyder and Stormo, in DNA and Protein Sequence Analysis, 1997, (Bishop and Rawlings, eds.), p 209-224, Oxford University Press, New York, N.Y.) and the like, or a combination thereof. In another aspect, comparing the putative cis-regulatory module to known cis-regulatory modules to further define SNP/indels occurrence rates is provided.

In one aspect, the decrease in frequency of SNPs is about 30% to about 50%. In a related aspect, the method includes calculating the ratio of SNPs in transcriptionally active sequences versus flanking sequences. In a further related aspect, the ratio determined is between about 0.1 to about 0.7.

In another aspect, the method includes calculating the ratio of indels of differing lengths in transcriptionally active sequences versus flanking sequences, where the length of the indels is about 1 to 5 nucleotides, about 6 to 10 nucleotides, about 11-15 nucleotides, about 16 to 20 nucleotides, or greater than about 21 nucleotides. In a related aspect, the ratio of indels of about 6 to 10 nucleotides is between about 0 to about 0.7.

In one aspect, genome wide computational maps of SNPs and indels may be constructed from the data generated by the disclosed method using closely related species, with reference to those species of interest (e.g., humans), to compute a moving window snp/indel intensity parameter as a function of position. For example, the basic idea is to slide a window across a query sequence and identify which region it matches best with each new position of the window. A query sequence is identified as a putative patch if it shows significant similarity to sequences identified in “selected genomic sequences.” The program accepts a query sequence and a background alignment, and allows the user to define the window-size, how window boundaries are determined, how gaps will be handled, and how absolute similarity and statistical significance will be indicated in program output.

The unlikelihood of the ratio given the local background can be computed, using, for example, a low order Markov model (see e.g., U.S. Pat. No. 6,772,069 and U.S. Pat. No. 6,470,277) for local background, in all regions of the genome, where unusual snp/indel ratio features of the appropriate size are stored as a look-up table that are accessed by comparing such features to the genes they are near. In a related aspect, computing a likelihood ratio via a first order Markov for the genome sequence is provided to represent the likelihood that a suppressed SNP/indel ratio will randomly occur in a sequence being analyzed.

US-2006-0141513 A1 also describes expression vectors comprising a putative cis-module operably linked to at least one reporter gene sequence. “Operably linked” is intended to mean that the cis-module sequence is linked to an expression cassette, such as a reporter gene sequence, in a manner that allows expression of the encoded (reporter gene) sequence. Reporter sequences are known in the art and are selected to determine transcriptional modulation in an appropriate host cell. (see, e.g., D. V. Goeddel (1990) Methods Enzymol. 185:3-7). It should be understood that the design of the expression vector may depend on such factors as the choice of the host cell to be transfected and/or the type of reporter desired to be expressed. Such reporter proteins include, but are not limited to, β-galactosidase, luciferase, chloramphenicol acetyltransferase, green fluorescent protein, secreted alkaline phosphatase, and the like.

Appropriate host cells for use with the method include bacteria, fungi, yeast, plant, insect, and animal cells, especially mammalian and human cells. Replication and inheritance systems include, but are not limited to, M13, Co1E 1, SV40, baculovirus, lambda, adenovirus, CEN ARS, 2 μm ARS, and the like.

Vectors can contain one or more replication and inheritance systems for cloning or expression, one or more markers for selection in the host, e.g., antibiotic resistance, and one or more expression cassettes. The inserted sequences of interest can be synthesized by standard methods, isolated from natural sources, or prepared as hybrids. Ligation of the sequences of interest can be carried out using established methods.

In one aspect, the method further includes operably linking the putative patch region to a reporter sequence in a vector and determining whether the reporter sequence is expressed in a host comprising the vector.

In another aspect, a canonical approach is used to computationally identify target cis-regulatory modules. The stripped sequences or putative patches are subjected to two forms of a priori analysis. They are first analyzed for statistical features indicative of putative cis-regulatory modules, and likely target regions are identified and displayed on sequence coordinates. These are regions where short sequence motifs appear in clusters (i.e., multiply, within a set distance with respect either to individual motifs, and/or several motifs in combination).

In one embodiment, two algorithms can be used: one statistical, the other heuristic (using artificial neural networks, see, e.g., Hatzigeorgiou, et al., 1996. Functional site prediction on the DNA sequence by artificial neural networks. In Proceedings of the IEEE International Joint Symposia on Intelligence and Systems, pp. 12-17. IEEE Computer Society Press, Los Alamitos, Calif.) to identify motifs of multiple putative transcription factor binding sites clustering within shorter (user defined) lengths of sequence such that the rate of occurrence of the clusters falls outside of statistical expectations. Exact patterns or user defined degrees of variability in the putative binding sites can be used.

The putative patches can be compared to the equivalent genomic sequence of related species, and then other species. For example, relevant sequences surrounding genes of interest in rat can be compared to that surrounding the same gene in, for example, mice and then to that surrounding the orthologous gene in humans. In one aspect, computational maps are generated from one or more closely related primate or murine species. In a related aspect, the primate is an ape, monkey, or human. In a further related aspect, cis-regulatory modules based on the primate derived computational map are compared to select genome sequences from non-primates and used to predict cis-regulatory modules in the non-primate sequences or vice versa.

Such comparisons are carried out using FAMILY RELATIONS program, or the like, and the results compared to the statistically selected regions of the same sequence, with particular weight given to interspecific conserved elements that also have the desired statistical features. In one aspect, a library of putative cis-regulatory modules is provided, where the modules are identified by the method as described.

In another embodiment, oligonucleotides, or longer fragments derived from conserved patch sequences described herein may be used as targets in a library/microarray (e.g., biochip) system. The microarray, for example, can be used to identify genetic variants, mutations, and polymorphisms. This information may be used to determine gene function, to understand the genetic basis of a disease, to diagnose disease, and to develop and monitor the activities of therapeutic or prophylactic agents. Preparation and use of microarrays have been described in WO 95/11995 to Chee et al.; Lockhart et al., Nature Biotechnology (1996) 14:1675-1680; Schena et al., Proc Natl Acad Sci USA (1996) 93:10614-10619; U.S. Pat. No. 6,015,702 to Lal et al.; Worley et al., Microarray Biochip Technology, (Schena, ed.), Biotechniques Book, Natick, Mass., (2000) pp. 65-86; Rogers et al., Anal Biochem (1999) 266(1):23-30; Head et al., Mol Cell Probes (1999) 13(2):81-7; Watson et al., Biol Psychiatry (2000) 48(12):1147-56.

In one aspect, microarrays containing arrays of conserved patch sequences can be used to identify mutations or polymorphisms in a population, including but not limited to, deletions, insertions, and mismatches. For example, mutations can be identified by: (i) placing cis-regulatory module polynucleotides of the present invention onto a biochip; (ii) taking a test sample and adding the sample to the biochip; (iii) determining if the test samples hybridize to the cis-regulatory module polynucleotides attached to the chip under various hybridization conditions (see, e.g., Chechetkin et al., J Biomol Struct Dyn (2000) 18(1):83-101). Alternatively microarray sequencing can be performed (see, e.g., Diamandis, Clin Chem (2000) 46(10):1523-1525).

In another aspect, methods of the present invention can be used to generate a database of transcription target site clusters comprising low SNP/indel ratios.

In another embodiment, a conserved patch sequence or cis-regulatory module, or a complementary sequence, or fragment thereof, can be used as a probe which is useful for mapping naturally occurring genomic sequences. The sequences may be mapped to a particular chromosome, to a specific region of a chromosome, or contig, to human artificial chromosome constructions (HACs), yeast artificial chromosomes (YACs), bacterial artificial chromosomes (BACs), bacterial PI constructions, or single chromosome cDNA libraries (see, e.g., Price, Blood Rev (1993) 7:127-134 and Trask, Trends Genet (1991) 7:149-154).

4. Vectors

The subject polynucleotide encoding the nuclear blocking sequence may be introduced into an organism, either as an extra chromosomal genetic element (such as a stably maintained extra chromosomal genetic element) or as a genetic element stably integrated into the host genome. There are numerous art-recognized methods for introducing exogenuous genetic elements (e.g., DNA constructs) into an organism (either individually cultured cells or as transgenic animal or both), especially the model organisms including S. cerevisea (budding yeast), S. pombe (fission yeast), C. elegans (worm), D. melanogaster (fruit fly), Danio rerio (zebrafish), Xenopus laevis (African clawed frog), Mus musculus (house mouse), Rattus norvegicus (Norway rat), Mesocricetus auratus (golden hamster), Monodelphis domestica (gray short-tailed opossum), Pan troglodytes (chimpanzee), Macaca mulatta (rhesus monkey), Macaca fascicularis (crab-eating macaque), Bos Taurus (cattle), Sus scrofa (pig), Equus caballus (horse), Canis familiaris (dog), Felis catus (cat), Gallus gallus (chicken), Arabidopsis thaliana, etc. Typically, they include introducing the construct into cells, embryos, gonads, etc., by way of microinjection, electroporation, transfection, infection, etc. Most, if not all the vectors described herein can be directly used or adapted to be used for such purposes using standard molecular biology techniques.

One aspect of the invention also relates to the use of a subject nucleic acid construct in “antisense therapy.” As used herein, “antisense therapy” refers to the use of oligonucleotide probes which specifically hybridizes (e.g., binds) under cellular conditions, with their cellular targets, e.g., pre-mRNA of a target gene in the nucleus of a target cell, so as to inhibit expression of the protein encoded by the target gene, e.g., by inhibiting transcription (splicing) and/or translation. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the target pre-mRNA.

General approaches to constructing oligomers useful in antisense therapy have been reviewed, for example, by van der Krol et al., Biotechniques 6: 958-976, 1988; and Stein et al., Cancer Res 48: 2659-2668, 1988. All incorporated herein by reference. Techniques described therein can all be adapted for use in the instant invention.

Accordingly, the nuclear blocking sequences of the invention are useful in therapeutic and research contexts. In therapeutic applications, the oligomers are utilized in a manner appropriate for antisense therapy in general. For such therapy, constructs encoding the oligomers of the invention can be formulated for a variety of loads of administration, including systemic and topical or localized administration. Techniques and formulations generally may be found in id Remmington's Pharmaceutical Sciences, Meade Publishing Co., Easton, Pa., and may include both human and vetinary formulations. For systemic administration, injection of the subject constructs is preferred, including intramuscular, intravenous, intraperitoneal, and subcutaneous injections. The constructs of the invention can be formulated in liquid solutions, preferably in physiologically compatible buffers such as Hank's solution or Ringer's solution. In addition, the subject constructs may also be formulated in solid form and redissolved or suspended immediately prior to use. Lyophilized forms are also included.

Likewise, the antisense constructs of the present invention, by antagonizing the normal biological activity of a target gene (by inhibiting its expression), can be used in the manipulation of tissue, e.g. tissue differentiation, both in vivo and in ex vivo tissue cultures, as well as in the treatment of pathological conditions associated with undesired expression of the target gene, such as in cancer treatment (e.g., down-regulation of oncogene expression), Cardiovascular applications (e.g., prevention of restenosis after angioplasty, coronary artery bypass graft, etc.), viral infection (e.g., Hepatitis C virus, West Nile virus, Influenza A virus, SARS virus, Dengue virus, Ebola virus, or Vesivirus, etc.).

The expression vectors of the invention comprises a nucleic acid sequence encoding the blocking sequence (e.g., antisense RNA) of a target gene, which nucleic acid sequence is operably linked to at least one transcriptional cis-regulatory module/sequence. Operably linked is intended to mean that the nucleic acid sequence is linked to the cis-regulatory sequence in a manner which allows expression of the nucleic acid sequence.

In certain embodiments, the regulatory sequences are art-recognized and are selected to direct expression of a subject antisense RNA. Accordingly, the term transcriptional cis-regulatory module/sequence includes promoters, enhancers and other expression control elements. Certain exemplary regulatory sequences are generally described in Goeddel; Gene Expression Technology: Methods in Enzymology 185, Academic Press, San Diego, Calif. (1990). For instance, any of a wide variety of expression control sequences—sequences that control the expression of a DNA sequence when operatively linked to it may be used in these vectors to express the subject nuclear blocking sequences, provided that they provide the desired temporal or spacial expression regulation, if any. Such useful expression control sequences, include, for example, cis-regulatory modules of any genes with a desirable temporal and/or spacial expression pattern. Such useful expression control sequences, depending on specific uses, may also include one or more generic regulatory elements such as the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof.

It should be understood that the design of the expression vector may depend on such factors as the choice of the host cell to be transformed and/or the types of protein desired to be transcriptionally regulated, and whether such regulation should be constitutive, or in a tissue-specific and/or developmental stage-specific manner, etc. Moreover, the vector's copy number, the ability to control that copy number and the expression of any other proteins encoded by the vector, such as antibiotic markers, fluorescent markers, or a second antisense RNA construct (either in cis or in trans) should also be considered.

Moreover, the nucleic acid constructs of the present invention can also be used as a part of a gene therapy protocol to deliver blocking nucleic acids (e.g., antisense RNA) of a target gene. Thus, another aspect of the invention features expression vectors for in vivo transfection and expression of an antisense RNA against a target gene in particular cell types, so as to abrogate the function of the target gene in a cell.

Expression constructs of the subject invention may be administered in any biologically effective carrier, e.g., any formulation or composition capable of effectively delivering the constructs to cells in vivo.

Exemplary approaches include insertion of the subject construct in viral vectors, including recombinant retroviruses, adenovirus, adeno-associated virus, and herpes simplex virus-1 or recombinant bacterial or eukaryotic plasmids. Viral vectors transfect cells directly; plasmid DNA can be delivered with the help of, for example, cationic liposomes (lipofectin) or derivatized (e.g., antibody-conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular gene construct provided for in vivo transduction of blocking sequence (e.g., antisense RNA) expression are also useful for in vitro transduction of cells.

A preferred approach for in vivo introduction of nucleic acid into a cell is by use of a viral vector containing nucleic acid, e.g. a DNA encoding an antisense RNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up the vector.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly A) with regard to the possibility of the spread of wild-type virus in the cell population. The development of specialized cell lines (termed “packaging cells”) which produce only replication-defective retroviruses has increased the utility of retroviruses for gene therapy and defective retroviruses are well characterized for use in gene transfer for gene therapy purposes (for a review see Miller, Blood 76: 271, 1990). Thus, recombinant retrovirus can be constructed in which part of the retroviral coding sequence (gag, pol, env) has been replaced by nucleic acid encoding a antisense RNA, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in, e.g., Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types. including epithelial cells, in vitro and/or in vivo (see for example Eglitis, et al. Science 230: 1395-1398, 1985; Danos and Mulligan, Proc. Natl. Acad. Sci. USA 85: 6460-6464, 1988; Wilson et al., Proc. Natl. Acad. Sci. USA 85: 3014-3018, 1988; Armentano et al., Proc. Natl. Acad Sci USA 87: 6141-6145, 1990; Huber et al., Proc. Natl. Acad. Sci USA 88: 8039-8043, 1991; Ferry et al., Proc. Natl. Acad. Sci. USA 88: 8377-8381, 1991; Chowdhury et al., Science 254: 1802-1805, 1991; van Beusechem et al., Proc. Natl. Acad. Sci. USA 89: 7640-7644, 1992; Kay et al., Human Gene Therapy 3: 641-647, 1992; Dai et al., Proc. Natl. Acad. Sci. USA 89: 10892-10895, 1992; Hwu et al., J Immunol. 150: 4104-4115, 1993; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al., PNAS 86: 9079-9083, 1989; Julan et al., J. Gen Virol 73: 3251-3255, 1992; and Goud et al., Virology 163: 251-254, 1983); or coupling cell surface receptor ligands to the viral env proteins (Neda et al., J. Biol Chem 266: 14143-14146, 1991). Coupling can be in the form of the chemical cross-linking with a protein or other variety receptor-ligand drug, as well as by generating fusion proteins (e.g. single-chain antibody/env fusion proteins). For example, agents which bind to β-cell receptors (either ligand or antibody) can be used to enhance infection of β-cells. To illustrate, derivatization of the viral particle with ligands for at least one of the glucagon-like peptide receptor (GLP), the sulfonylurea receptor, the galanin receptor, or antibodies against β-cell antigens, such as GAD65. This technique, while useful to limit or otherwise direct the infection to pancreatic tissue, can also be used to convert an ecotropic vector in to an amphotropic vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al., BioTechniques 6: 616, 1988; Rosenfeld et al., Science 252: 431-434, 1991; and Rosenfeld et al., Cell 68: 143-155, 1992. Suitable adenoviral vectors derived from the adenovirus strain Ad type 5 d1324 or other strains of adenovirus (e.g., Ad2, Ad3, Ad7 etc.) are well known to those skilled in the art. The virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57: 267, 1986). Most replication-defective adenoviral vectors currently in use and therefore favored by the present invention are deleted for all or parts of the viral E1 and E3 genes but retain as much as 80% of the adenoviral genetic material (see, e.g., Jones et al., Cell 16: 683, 1979; Berkner et al., supra; and Graham et al. in Methods in Molecular Biology, E. J. Murray, Ed. (Humana, Clifton, N.J., 1991) vol. 7. pp. 109-127). Expression of the inserted antisense RNA can be under control of, for example, the EIA promoter, the major late promoter (MLP) and associated leader sequences, the E3 promoter, or exogenously added promoter or cis-regulatory sequences that provides desired expression pattern.

Yet another viral vector system useful for delivery of the subject antisense RNA is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158: 97-129, 1992). It is also one of the few viruses that may integrate its DNA into non-dividing cells, and exhibits a high frequency of stable integration (see for example Flotte et al., Am. J. Respir. Cell. Mol. Biol. 7: 349-356, 1992; Samulski et al., J. Virol. 63: 3822-3828, 1989; and McLaughlin et al., J. Virol. 62: 1963-1973, 1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5: 3251-3260, 1985 can be used to introduce antisense sequence into cells. A variety of nucleic acids have been introduced into different cell types using AAV vectors (see for example Hermonat et al., Proc. Natl. Acad. Sci. USA 81: 6466-6470, 1984; Tratschin et al., Mol. Cell. Biol. 4: 2072-2081, 1985; Wondisford et al., Mol. Endocrinol. 2: 32-39, 1988; Tratschin et al., J. Virol. 51: 611-619, 1984; and Flotte et al., J. Biol. Chem. 268: 3781-3790, 1993).

In addition to viral transfer methods, such as those illustrated above, non-viral methods can also be employed to cause expression of a subject antisense sequence in the tissue of an animal. Most nonviral methods of gene transfer rely on normal mechanisms used by mammalian cells for the uptake and intracellular transport of macromolecules. In preferred embodiments. non-viral gene delivery systems of the present invention rely on endocytic pathways for the uptake of the subject antisense sequences by the targeted cell. Exemplary gene delivery systems of this type include liposomal derived systems, poly-lysine conjugates, and artificial viral envelopes.

In a representative embodiment, a therapeutic antisense construct can be entrapped in liposomes bearing positive charges on their surface (e.g., lipofectins) and (optionally) which are tagged with antibodies or ligands for pancreatic cell surface antigens (Mizuno et al., No Shinkei Geka 20: 547-551, 1992; PCT publication WO91/06309; Japanese patent application 1047381; and European patent publication EP-A-43075). For example, lipofection of β-cells can be carried out using liposomes tagged with monoclonal antibodies against, for example, the GAD65 antigen, or any other cell surface antigen present on these pancreatic cells. Alternatively, liposomes can be derivative with such receptor ligands glimepiride, glibenclamide or other sulfonylurea drug.

In clinical settings, the gene delivery systems for therapeutic antisense constructs can be introduced into a patient (or non-human animal) by any of a number of methods, each of which is familiar in the art. For instance, a pharmaceutical preparation of the gene delivery system can be introduced systemically, e.g. by intravenous injection, and specific transduction of the antisense in the target cells occurs predominantly from specificity of transfection provided by the gene delivery vehicle, cell-type or tissue-type expression due to the transcriptional regulatory sequences controlling expression of the construct, or a combination thereof. In other embodiments, initial delivery of the recombinant gene is more limited with introduction into the animal being quite localized. For example, the gene delivery vehicle can be introduced into the pancreas by catheter (see U.S. Pat. No. 5,328,470), by stereotactic injection (e.g. Chen et al., PNAS 91: 3054-3057, 1994), or by electroporation during a partial pancreatectomy (Dev et al., Cancer Treat Rev 20: 105-115, 1994).

The pharmaceutical preparation of the gene therapy construct can consist essentially of the gene delivery system in an acceptable diluent, or can comprise a slow release matrix (such as controlled-release matrix or coating) in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

The vectors of this invention can be delivered into host cells via a variety of methods, including but not limited to, liposome fusion (transposomes), infection by viral vectors, and routine nucleic acid transfection methods such as electroporation, calcium phosphate precipitation and microinjection. In some embodiments, the vectors are integrated into the genome of a transgenic animal (e.g., a mouse, a rabbit, a hamster, or a nonhuman primate). Diseased or disease-prone cells containing these vectors can be used as a model system to study the development, maintenance, or progression of a disease that is affected by the presence or absence of the nuclear blocking sequence.

Expression of the nuclear blocking sequence introduced into a target cell may be confirmed by art-recognized techniques, such as RT-PCR, Northern blotting using a nucleic acid probe, etc. For cell lines that are more difficult to transfect, more extracted RNA can be used for analyses, optionally coupled with exposing the film longer. Once expression of the nuclear blocking sequence is confirmed, the DNA construct can then be tested for inhibition efficacy against a cotransfected construct encoding the target protein or directly against an endogenous target. In the latter case, one preferably should have a clear idea or at least an estimate of transfection efficiency and of the half-life of the target protein before performing the experiment.

5. Pharmaceutical Use and Methods of Administration

In one aspect, the invention provides a method of administering any of the compositions described herein (e.g., the constructs/vectors comprising a subject nuclear blocking sequence) to a subject. When administered, the compositions are applied in a therapeutically effective, pharmaceutically acceptable amount as a pharmaceutically acceptable formulation.

As used herein, the term “pharmaceutically acceptable” is given its ordinary meaning. Pharmaceutically acceptable compounds are generally compatible with other materials of the formulation and are not generally deleterious to the subject. Any of the compositions of the present invention may be administered to the subject in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result, i.e., that amount which is capable of at least partially preventing, reversing, reducing, decreasing, ameliorating, or otherwise suppressing the particular condition being treated. A therapeutically effective amount can be determined on an individual basis and will be based, at least in part, on consideration of the species of mammal, the mammal's age, sex, size, and health; the compound and/or composition used, the type of delivery system used; the time of administration relative to the severity of the disease; and whether a single, multiple, or controlled-release dose regiment is employed. A therapeutically effective amount can be determined by one of ordinary skill in the art employing such factors and using no more than routine experimentation.

The terms “treat,” “treated,” “treating,” and the like, when used herein, refer to administration of the systems and methods of the invention to a subject, which may, for example, increase the resistance of the subject to development or further development of cancers, to administration of the composition in order to eliminate or at least control a cancer or a infectious disease, and/or to reduce the severity of the cancer or infectious disease, or symptoms thereof. Such terms also include prevention of disease/condition in, for example, subjects/individuals predisposed to such diseases/conditions, or at high risk of developing such diseases/conditions.

When administered to a subject, effective amounts will depend on the particular condition being treated and the desired outcome. A therapeutically effective dose may be determined by those of ordinary skill in the art, for instance, employing factors such as those further described below and using no more than routine experimentation.

In administering the systems and methods of the invention to a subject, dosing amounts, dosing schedules, routes of administration, and the like may be selected so as to affect known activities of these systems and methods. Dosage may be adjusted appropriately to achieve desired drug levels, local or systemic, depending upon the mode of administration. The doses may be given in one or several administrations per day. As one example, if daily doses are required, daily doses may be from about 0.01 mg/kg/day to about 1000 mg/kg/day, and in some embodiments, from about 0.1 to about 100 mg/kg/day or from about 1 mg/kg/day to about 10 mg/kg/day. Parental administration, in some cases, may be from one to several orders of magnitude lower dose per day, as compared to oral doses. For example, the dosage of an active compound when parentally administered may be between about 0.1 micrograms/kg/day to about 10 mg/kg/day, and in some embodiments, from about 1 microgram/kg/day to about 1 mg/kg/day or from about 0.01 mg/kg/day to about 0.1 mg/kg/day.

In some embodiments, the concentration of the active compound(s), if administered systemically, is at a dose of about 1.0 mg to about 2000 mg for an adult of 70 kg body weight, per day. In other embodiments, the dose is about 10 mg to about 1000 mg/70 kg/day. In yet other embodiments, the dose is about 100 mg to about 500 mg/70 kg/day. Preferably, the concentration, if applied topically, is about 0.1 mg to about 500 mg/gm of ointment or other base, more preferably about 1.0 mg to about 100 mg/gm of base, and most preferably, about 30 mg to about 70 mg/gm of base. The specific concentration partially depends upon the particular composition used, as some are more effective than others. The dosage concentration of the composition actually administered is dependent at least in part upon the particular physiological response being treated, the final concentration of composition that is desired at the site of action, the method of administration, the efficacy of the particular composition, the longevity of the particular composition, and the timing of administration relative to the severity of the disease. Preferably, the dosage form is such that it does not substantially deleteriously affect the mammal. The dosage can be determined by one of ordinary skill in the art employing such factors and using no more than routine experimentation.

In the event that the response of a particular subject is insufficient at such doses, even higher doses (or effectively higher doses by a different, more localized delivery route) may be employed to the extent that subject tolerance permits. Multiple doses per day are also contemplated in some cases to achieve appropriate systemic levels within the subject or within the active site of the subject. In some cases, dosing amounts, dosing schedules, routes of administration, and the like may be selected as described herein, whereby therapeutically effective levels for the treatment of cancer are provided.

In certain embodiments where cancers are being treated, a composition of the invention may be administered to a subject who has a family history of cancer, or to a subject who has a genetic predisposition for cancer. In other embodiments, the composition is administered to a subject who has reached a particular age, or to a subject more likely to get cancer. In yet other embodiments, the compositions is administered to subjects who exhibit symptoms of cancer (e.g., early or advanced). In still other embodiments, the composition may be administered to a subject as a preventive measure. In some embodiments, the inventive composition may be administered to a subject based on demographics or epidemiological studies, or to a subject in a particular field or career.

Administration of a composition of the invention to a subject may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the active compound(s) of the composition within the subject without causing clinically unacceptable adverse effects.

Any medically acceptable method may be used to administer a composition to the subject. The administration may be localized (i.e., to a particular region, physiological system, tissue, organ, or cell type) or systemic, depending on the condition being treated. For example, the composition may be administered orally, vaginally, rectally, buccally, pulmonary, topically, nasally, transdermally, through parenteral injection or implantation, via surgical administration, or any other method of administration where suitable access to a target is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound. Other oral compositions suitable for use with the invention include solutions or suspensions in aqueous or non-aqueous liquids such as a syrup, an elixir, or an emulsion. In another set of embodiments, the composition may be used to fortify a food or a beverage.

Injections can be e.g., intravenous, intradermal, subcutaneous, intramuscular, or interperitoneal. The composition can be injected interdermally for treatment or prevention of infectious disease, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

In general, the compositions of the invention may be delivered using a bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In certain embodiments of the invention, the administration of the composition of the invention may be designed so as to result in sequential exposures to the composition over a certain time period, for example, hours, days, weeks, months or years. This may be accomplished, for example, by repeated administrations of a composition of the invention by one of the methods described above, or by a sustained or controlled release delivery system in which the composition is delivered over a prolonged period without repeated administrations. Administration of the composition using such a delivery system may be, for example, by oral dosage forms, bolus injections, transdermal patches or subcutaneous implants. Maintaining a substantially constant concentration of the composition may be preferred in some cases.

Other delivery systems suitable for use with the present invention include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing drugs are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some embodiments of the invention. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

In some embodiments, the compositions of the invention may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. Those of skill in the art can readily determine the various parameters for preparing and formulating the compositions of the invention without resort to undue experimentation.

In some embodiments, the present invention includes the step of forming a composition of the invention by bringing an active compound into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product.

6. Exemplary Uses

Methods of the invention have broad uses in any medical and research settings where it is desirable to modulate the expression of a target gene. The following are merely illustrative uses, which should not be construed to be limiting in any respect.

Drug Target Validation

Good drugs are potent and specific; that is, ideally, they must have strong effects on a specific biological pathway or tissue (such as the disease tissue), while having minimal effects on all other pathways or all other tissues (e.g., healthy tissues). Confirmation that a compound inhibits the intended target (drug target validation) and the identification of undesirable secondary effects are among the main challenges in developing new drugs.

Modern drug screening typically requires tremendous amounts of time and financial resources. Ideally, before even committing to such an extensive drug development program to identify a drug, one would like to know whether the intended drug target would even make a good target for treating a disease. That is, whether antagonizing the function of the intended target (such as a disease-associated oncogene or survival gene), would be sufficient/effective to treat the disease, and whether such treatment would bear an acceptable risk or side effect. For example, if a cancer is determined to be caused by an activating mutation in the Ras pathway, or caused by abnormal activity of a survival gene such as Bcl-2, the subject system can be used to generate animal models for drug target validation. Specifically, one can generate a transgenic mouse with the subject nuclear blocking sequence, with the sequence targeting a gene that is a potential drug target (i.e., Ras or Bcl-2 in this example). Tumors with various initiating lesions can then be made in the mouse, and the nuclear blocking sequence can then be switched on or off (or turned up or down) in the tumor in a tissue-specific and/or developmental stage-specific manner (if, for example, a tet-ON regulator is used). Such nuclear blocking sequence expression mimicks the action of a drug that would interfere with that target. If knocking down the target gene is effective to reverse or stall the course of the disease, the target gene is a valid target.

Optionally, the nuclear blocking sequence transgene can be switched on in a number of tissues or organs, or even in the whole organism, in order to verify the potential side effects of the (yet to be identified) drug on other healthy tissues/organs.

Thus another aspect of the invention provides an animal useful for drug target validation, comprising a germline transgene encompassing the subject artificial nucleic acid, which transcription is driven by a subject cis-regulatory element (e.g., those comprising a Pol II promoter). The expression of the encoded nuclear blocking sequence (such as an antisense RNA) leads to a decreased or eliminated expression of the candidate drug target. Optionally, the nuclear blocking sequence is expressed in an inducible, reversible, and/or tissue-specific manner.

In a related aspect, the invention provides a method for drug target validation, comprising antagonizing the function of a candidate drug target (gene) using a subject cell or animal (e.g., a transgenic animal) encompassing the subject artificial nucleic acid, either in vitro or in vivo, and assessing the ability of the encoded nuclear blocking sequence to reverse or stall the disease progress or a particular phenotype associated with a pathological condition. Optionally, the method further comprises assessing any side effects of inhibiting the function of the target gene on one or more healthy organs/tissues.

Animal Disease Model

The subject nucleic acid constructs enables one to switch on or off a target gene or certain target genes (e.g., by using crossing different lines of transgenic animals to generate multiple-transgenic animals) inducibly, reversibly, and/or in a tissue-specific manner. This would faciliate conditional knock-out or turning-on of any target gene(s) in a tissue-specific manner, and/or during a specific developmental stage (e.g., embryonic, fetal, neonatal, postnatal, adult, etc.). Animals bearing such transgenes may be treated, such as by providing a tet analog in drinking water, to turn on or off certain genes to allow certain diseases to develop/manifest. Such system and methods are particularly useful, for example, to analyze the role of any known or suspected tumor suppressor genes in the maintenance of immortalized or transformed states, and in continued tumor growth in vivo.

In certain embodiments, the extent of gene knock-down may be controlled to achieve a desired level of gene expression. Such animals or cell (healthy or diseased) may be used to study disease progress, response to certain treatment, and/or screening for drug leads.

The ability of the subject system to use the nuclear blocking sequence (such as an integrated genomic copy of the nuclear blocking sequence) to control gene expression is particularly valuable for complex library screening.

### EXAMPLES

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

Applicants have developed a system to sidestep problems of gene regulation, by regulating the spatial and temporal effects of targeted gene knockdown. The method uses DNA expression constructs where time- and tissue-specific cis-regulatory sequences drive transcription of splice-blocking antisense oligomers. Using this method, Applicants have achieved spatially- and temporally-restricted knockdown of two test genes, Ets1 and Alx1, which are critical for proper skeletogenesis in the purple sea urchin Strongylocentrotus purpuratus. Similar approach may be generally used in any eukaryotic organisms.

Briefly, Applicants used early-acting enhancer sequences derived from the promoter for the Tbr gene to effect the specific expression of the encoded antisense sequences in the skeletogenic cells. Applicants targeted either Ets1 or Alx1 transcripts for destruction prior to the ingression of skeletogenic cells. Applicants found that this perturbation successfully blocked normal migration patterns as observed in control embryos. In contrast, using instead a late-acting enhancer from the Sm30 gene, which only operates at times subsequent to skeletogenic cell ingression, Applicants observed normal cell ingression. Critically, however, these same Sm30 test embryos showed defects in later skeletogenesis activities. This reflected the downregulation of either Alx1 or Ets1 only after migration had occurred and when Sm30 promoter elements were activated, thereby demonstrating the strict control of antisense transgene expression.

In addition, for this method to work, no in-depth knowledge of promoter sequences is required. All that is used is basic means of transgenesis. Thus the methods and systems of the invention can achieve spatial/temporal control of gene knockdown, and can therefore be applied to a wide range of applications in virtually any eukaryotic system.

The following describes certain details of the exemplary non-limiting experiments.

To demonstrate spatial and temporal regulation of targeted gene knockdown using the subject systems and methods, Applicants used the model developmental organism, the purple sea urchin Strongylocentrotus purpuratus. Delivery of nucleic acid vectors in this organism was easily achieved by microinjection into the fertilized eggs of sea urchins according to standard methods.

Specifically, adult Strongylocentrotus purpuratus were collected along the Southern California coast and maintained in 12° C. seawater. Gametes were harvested and eggs rinsed for one minute in 1 mM citric acid seawater, and were subsequently placed in seawater containing 300 mg/mL of para-aminobenzoic acid. Approximately 1500 molecules of desired DNA construct (or 450 molecules for large vectors such as BACs) were injected along with a 6-fold molar excess of HindIII-digested carrier sea urchin DNA per egg, in a 4 pL volume of 0.12 M KCl. The DNA constructs, typically PCR products, were injected into eggs immediately following fertilization.

The antisense construct used in these experiments contains three main elements (FIG. 1): cis-regulatory sequence from a given gene (or “driver gene”); antisense sequence targeting a splice junction of a gene (“target gene”); and a stabilizing sequence, such as that taken from the SV40 3′-UTR. The driver gene is a gene expressed at the desired time and place for knocking out target gene function. The identity of the driver gene and the target gene may be the same or different. The cis-regulatory sequence of the driver gene can be virtually any length, and precise knowledge of the regulatory elements is not necessary. For practical purposes, regions under 4-6 kb in length are desirable for certain methods, such as for designing constructs by the fusion PCR methods described below.

FIG. 2 describes two processes that may be used for targeted antisense vector construction.

According to one embodiment of the invention, PCR (preferably conducted using the High-fidelity PCR kit, Roche) was used to amplify the desired cis-acting sequence, using a right (downstream) primer with a universal adaptamer tail. This PCR product was called the “driver PCR fragment.” In parallel, an oligomer was synthesized (Integrated DNA Technologies, Coralville, Iowa, the “antisense oligo”). This was designed to comprise the following sequence elements, in 5′ to 3′ order: the reverse complement SV40 poly-adenylation sequence; the sense target site; and a universal adaptamer sequence (optional).

Fusion PCR was then performed to combine the driver PCR fragment and antisense oligo as follows: equal molar amounts of driver PCR fragment (desalted) and target oligo were added to 1×PCR mix containing buffer, dNTPs and enzyme to a final volume of 100 μL. The resulting reaction mix was then distributed among 10 PCR tubes and placed in a gradient thermal cycler with the following cycling protocol: 95° C. for 20 seconds, 54° C.-62° C. for 30 seconds, 68° C. for an appropriate time according to the length of the driver PCR fragment (e.g., ˜1 min. per kb) for 12 to 15 cycles. Note that no additional primers were added for this reaction. The target oligo and driver PCR fragment in essence “prime” each other by annealing at the universal adaptamer sequence. A secondary reaction mix, this time containing outside primers, was added immediately following completion of the primary reaction, and PCR was performed again with the same protocol for 25-30 additional cycles. PCR products were desalted by Qiagen Qiaquick columns and sequenced.

Though the instant application provides methods to identify driver gene cis-regulatory sequences to the required level of resolution or better, Applicants found that it is indeed possible to construct spatially- and temporally-controlled targeting vectors without any prior knowledge of cis-regulatory sequences at all. To achieve this, Applicants used BAC clones containing vast amounts of genomic DNA surrounding the driver gene coding region as the control agent (FIG. 2, bottom panel). First, a BAC clone for the driver gene was identified, and at a minimum, a small amount of sequence surrounding the start of transcription was determined. A cassette containing the target antisense sequence and a bacterial kanamycin resistance gene flanked on either side by ˜45 bp of driver gene sequence was made by fusion PCR methods (similar to those described above). Homologous recombination was induced by standard methods in E. coli. Bacteria transformed with the driver BAC clone and the recombination cassette, and clones selected for kanamycin resistance. The resulting BAC recombinants were harvested, checked by sequencing, and prepared for injection by linearization with either Not I or Asc I restriction digest, followed by drop dialysis. By this approach, one can still utilize the method of temporally-/spatially-regulated gene knockdown in the complete absence of information regarding the particular cis-acting genomic sequences.

Next, Applicants examined the temporal functions of two test genes expressed in the skeletogenic cell lineage: Alx1 and Ets1. In prior experiments, morpholino-substituted oligonucleotide (MASO) targeting either Alx1 and Ets1 indicated an early essential function of each of these genes in skeletogenic cell ingression. Skeletogenic cells normally migrate into the blastocoelar space around 24-hr post-fertilization. MASO against either Alx1 or Ets1 blocks such migration. Given that normal expression in the skeletogenic cells of both Alx1 and Ets1 extends well-beyond migration, Applicants reasoned that Alx1 and Ets1 have functions in skeletogenesis past initial ingression. Unfortunately, gene knockdown by MASO, with its lack of regulatory control over antisense expression, would not be useful for understanding these functions.

The methods of the invention addresses the problem, in that the methods of the invention are well-suited to study the later functions of Alx1 and Ets1. To this end, Applicants have designed four classes of temporally- and spatially-regulated antisense constructs using one of two drivers expressed exclusively in skeletogenic cells: an early, pre- and post-ingression driver (the Tbr gene promoter); or a late-only, post-ingression driver (the Sm30 gene). With these drivers, Applicants targeted splice junctions in either the Alx1 or Ets1 genes.

It is known that zygotic expression of the Tbr gene starts around 7-hr post-fertilization, and it continues on through skeletonization. In contrast, the Sm30 promoter is not active until after 30-hr. FIG. 3 summarizes the assays used and the timing of expression of the driver genes. The classes of targeting vectors are described below:

I. Tbr promoter driving antisense oligomer targeting Alx1 (“Tbr->Alx1 antisense”)

II. Tbr promoter driving antisense oligomer targeting Ets1 (“Tbr->Ets1 antisense”)

III. Sm30 promoter driving antisense oligomer targeting Alx1 (“Sm30->Alx1 antisense”)

IV. Sm30 promoter driving antisense oligomer targeting Ets1 (“Sm30->Ets1 antisense”)

V. Tbr promoter driving sense oligomer targeting Alx1 (“Tbr->Alx1 sense”)

VI. Tbr promoter driving sense oligomer targeting Ets1 (“Tbr->Ets1 sense”)

VII. Sm30 promoter driving sense oligomer targeting Alx1 (“Sm30->Alx1 sense”)

VIII. Sm30 promoter driving sense oligomer targeting Ets1 (“Sm30->Ets1 sense”)

The gene name to the left of the “->” symbol indicates which gene is the driver and to the right the target. Classes I and II were made both with the standard PCR-directed method using a 1 kb piece of the Tbr promoter previously found to contain the relevant regulatory information, and by using the Tbr BAC to drive antisense oligomer expression. Classes III and IV were made with the PCR fusion method only. For negative controls, Applicants constructed the same classes of vectors, but with sense oligomers being expressed in place of the antisense ones (Classes V through VIII, referred to as “sense controls”).

Two technical aspects of sea urchin biology provide both a problem and a solution for proceeding to test knockdown constructs.

The problem arises as a result of mosaic incorporation of introduced linear DNA molecules, i.e., not all cells of the recipient embryo harbor the exogenous DNA. Therefore, a marker indicating which cells have incorporated the antisense targets is needed.

The solution is provided by the fact that linear DNA molecules incorporate in large concatenates (on the order of 10² molecules). Thus multiple species of co-injected linear DNA constructs incorporate together. For the present purposes, one of those constructs can be used as a marker of injection and incorporation. For example, Applicants used the Tbr BAC-GFP recombinant for such purposes. The Tbr BAC-GFP is a Tbr BAC clone with the coding region for the green fluorescent protein (GFP) knocked into the start of translation of the Tbr gene. Tbr has zygotic expression in the cells of the skeletogenic lineage from as early as 7-hr post-fertilization, and it continues through skeletogenesis (>48 hr post-fertilization). This is reflected in GFP expression in skeletogenic cells in roughly 40% of the embryos injected with the Tbr BAC-GFP construct, itself reflecting the rate of mosaic incorporation. Only, and all, cells expressing GFP carry the antisense vectors. All data are therefore expressed as a proportion of embryos expressing GFP.

Thus the injection solutions included both the antisense-expressing construct (either PCR product or linearized BAC vector), and linearized Tbr BAC-GFP as a marker of incorporations. Excess Hind III-digested sea urchin genomic DNA may also be (and was indeed) used as a carrier in a 0.12 M KCl solution. The target injection volume is about 4 pL in these experiments. This is roughly equivalent to about 1,500 molecules of antisense constructs, or about 450 molecules of BAC delivered per egg. Applicants then followed the injected embryos by observing GFP expression at 24-hr post-fertilization, a time when skeletogenic cell ingression in unperturbed embryos is at or near completion, and again at 48-hr post-fertilization, after normal skeleton mineralization has commenced. For negative controls, Applicants used sense controls in place of antisense-expressing constructs.

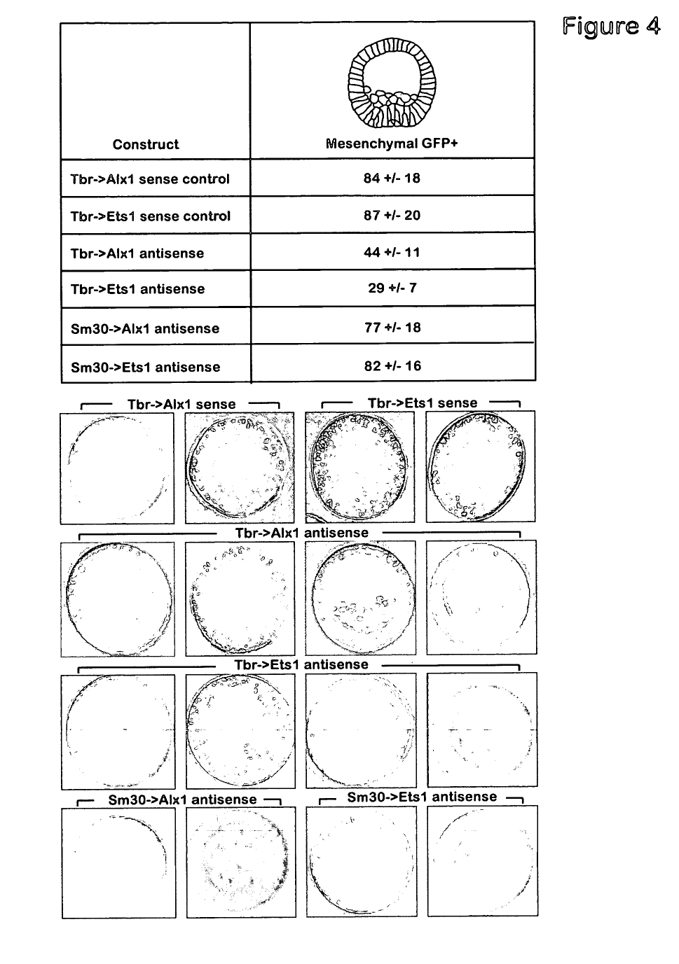

Applicants found that at 24-hr post-injection, GFP-labeled cells failed to ingress significantly more often in embryos harboring Tbr->Alx1 antisense or Thr->Ets1 antisense than in sense controls (FIG. 4). Applicants found nearly identical results when using a Tbr BAC-based vector to drive antisense oligomer expression, thereby demonstrating that this method can be used for spatially- and temporally-directed gene knockdown with no previous knowledge of driver-gene promoter elements.

By contrast, the Sm30-driven antisense constructs (Sm30->Alx1 antisense or Sm30->Ets1 antisense) showed no effect on migration (FIG. 4). The same proportion of embryos injected with either Sm30-driven construct showed complete migration as in the controls. Sm30-driven constructs therefore do not have any discernible effects prior to the time Sm30 gene expression starts.

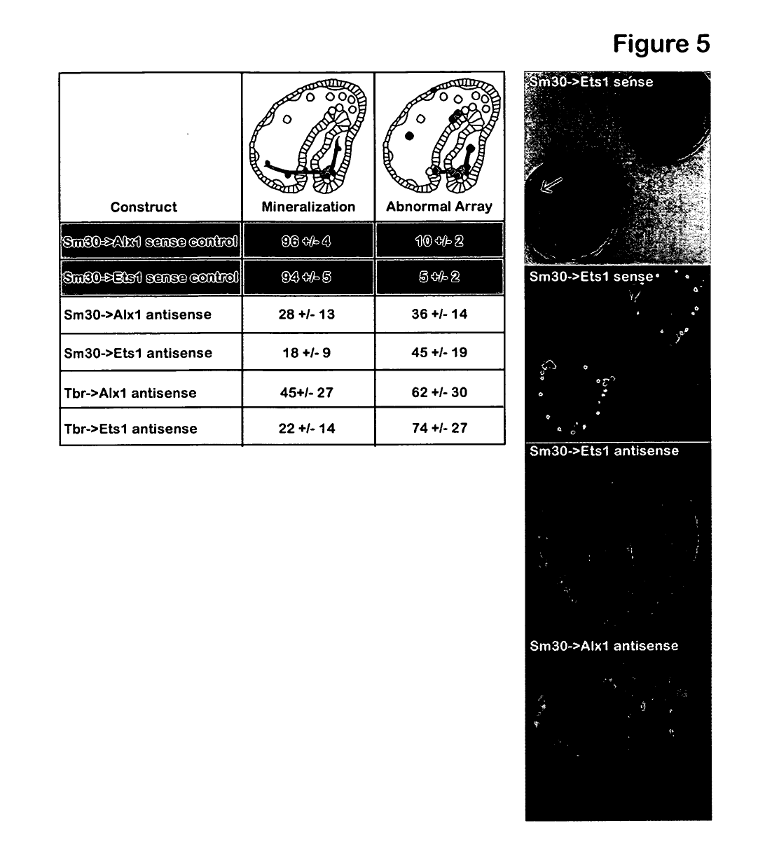

Of central importance to the present technology, however, the Sm30-driven antisense constructs do inhibit skeletonization at time points after Sm30 gene activation begins. The skeletonization phenotype analyzed at 48-hr had two related aspects. The first is the array that skeletonogenic mesenchymal cells form. Around 30-hr post-fertilization (also about the same time as the Sm30 promoter becomes active), the skeletonogenic mesenchymal cells form a syncytium, thus connecting the cytoplasms of all these cells, after which they migrate to discrete regions within the blastocoel. This array has a very characteristic look (see FIG. 5, right panel). Since GFP can freely diffuse within the syncytium, it is easily identified. Applicants scored normal versus abnormal array formation. As expected, the Tbr-driven constructs showed strong effects on array formation: a portion of the skeletonogenic cells in these embryos failed to migrate in the first place. Critically, the Sm30-driven antisense constructs also showed strong effects (FIG. 5). Abnormal arrays were stunted at best, but more often unidentifiable as arrays in most recipient embryos.

The second part of the phenotype is mineralization. Even embryos which do not form a discernible array may still form nuclei of mineralization, so the lack of mineralization can be more informative as to the state of the skeletonogenesis program as a whole.

Birefringence allows simple detection of mineralized spicules under polarized light. Applicants scored for mineralization at 48-hr post-fertilization, by which time mineralization is underway in virtually all control embryos (FIG. 5). In a strong demonstration of the potency of this technology, injection of Sm30->Alx1 antisense or Sm30->Ets1 antisense greatly inhibited mineralization. Introduction of Tbr->Alx1 antisense or Tbr->Ets1 antisense performed similarly, while embryos treated with sense controls displayed mineralization in virtually every instance. The block of mineralization by Sm30-regulated vectors represents not only a dramatic phenotype in its own right, but a stark contrast to the lack of effect for Sm30-driven constructs at the earlier time point. This stands as testament to the exquisite temporal and spatial control exercised by driver gene regulatory sequences.

In summary, the technology presented here allows for the coupling of this precise regulatory control with gene knockdown methods, a combination of tremendous advantage for functional biology studies in a virtually unlimited number of systems.

The practice of aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). All patents, patent applications and references cited herein are incorporated in their entirety by reference.

Equivalents

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

All publications mentioned herein are incorporated herein by reference, for the purpose of describing and disclosing the subject components of the invention that are described in the publications, which components might be used in connection with the presently described invention.

1. A method for inhibiting the expression of a target gene in an organism, the method comprising:
(1) providing a nucleic acid construct comprising a polynucleotide sequence encoding a nuclear blocking sequence of the target gene, said polynucleotide sequence is operably linked to a cis-regulatory module which directs the temporal- and/or spacial-expression of said nuclear blocking sequence;
(2) introducing said nucleic acid construct into the organism to allow the expression of said nuclear blocking sequence, thereby inhibiting the expression of the target gene in the organism.

2. The method of claim 1, wherein said nuclear blocking sequence binds in the nucleus to a portion of the target gene transcript.

3. (canceled)

4. The method of claim 1, said target gene comprises one or more introns, and said nuclear blocking sequence inhibits splicing of the target gene transcript.

5. The method of claim 4, wherein the cis-regulatory module is a regulatory sequence controlling the spacial- and/or temporal-expression of the target gene.

6. The method of claim 4, wherein the cis-regulatory module is a regulatory sequence controlling the spacial- and/or temporal-expression of a second gene different from the target gene.

7. The method of claim 4, wherein the nuclear blocking sequence is an antisense RNA complementary to a portion of the target gene transcript.

8. (canceled)

9. The method of claim 7, wherein the portion of the target gene transcript spans the upstream splice junction of the target gene.

10. The method of claim 9, wherein the splice junction is an exon-intron junction or a splice donor.

11. The method of claim 10, wherein the splice junction spans the first exon or the first intron.

12. The method of claim 9, wherein the splice junction is an intron-exon junction or a splice acceptor.

13. The method of claim 7, wherein the length of the antisense RNA is about 25-40 bases.

14. (canceled)

15. The method of claim 7, wherein the antisense RNA binds to the target gene transcript in the nucleus to inhibit splicing.

16. The method of claim 4, wherein the nucleic acid construct is stably integrated into the genome of at least one cell of the organism.

17. (canceled)

18. The method of claim 4, wherein the organism is a eukaryote.

19. The method of claim 18, wherein the eukaryote is a plant, a mammal, or an echinoderm.

20. The method of claim 4, wherein the organism is a cell.

21. The method of claim 4, wherein the nucleic acid construct inhibits the expression of the target gene in vitro.

22. The method of claim 4, wherein the nucleic acid construct inhibits the expression of the target gene in vivo.

23-26. (canceled)

27. The method of claim 4, wherein the cis-regulatory module comprises an inducible promoter, a tissue-specific promoter, and/or a developmental stage-specific promoter.

28-30. (canceled)

31. A nucleic acid construct comprising a polynucleotide sequence encoding a nuclear blocking sequence of a target gene in an organism,
wherein said polynucleotide sequence is operably linked to a cis-regulatory module which directs the temporal-, spacial-, and/or inducible-expression of said nuclear blocking sequence upon introducing the nucleic acid construct into the organism, and
wherein said target gene comprises one or more introns, and said nuclear blocking sequence inhibits splicing of the target gene transcript.

32. An organism comprising the nucleic acid construct of claim 31.

33-36. (canceled)

37. A method for treating a gene-mediated disease, comprising introducing into an individual having the disease a construct according to claim 31, where the nuclear blocking sequence is specific for the gene mediating the disease.

38. (canceled)

39. A method of validating a candidate gene as a potential target for treating a disease, comprising:
(1) introducing a construct according to claim 31 into a cell associated with the disease, wherein the nuclear blocking sequence is specific for the candidate gene;
(2) assessing the effect of inhibiting the expression of the candidate gene on one or more disease-associated phenotypes;
wherein a positive effect on at least one disease-associated phenotype is indicative that the candidate gene is a potential target for treating the disease.

40. The method of claim 39, wherein the cis-regulatory module comprises an inducible promoter.

41. The method of claim 39, wherein the candidate gene is over-expressed or abnormally active in disease cells or tissues.

42-48. (canceled)
